(19) United States
(12) Patent Application Publication
(10) Pub. No.: US 2016/0317162 A1
(43) Pub. Date: Nov. 3, 2016

(54) METHODS AND SYSTEMS FOR LIGAMENT REPAIR

(71) Applicant: Tenjin LLC, Brazoria, TX (US)

(72) Inventors: Christopher P. DOUGHERTY, Rogers, AR (US); Gary R. HEISLER, Brazoria, TX (US); Robert A. VAN WYK, St. Pete Beach, FL (US)

(73) Assignee: Tenjin LLC, Brazoria, TX (US)

(21) Appl. No.: 15/142,120

(22) Filed: Apr. 29, 2016

Related U.S. Application Data
(60) Provisional application No. 62/179,167, filed on Apr. 29, 2015.
(60) Provisional application No. 62/284,479, filed on Oct. 1, 2015.
(60) Provisional application No. 62/284,714, filed on Oct. 7, 2015.

(51) Int. Cl. A61B 17/16

(52) U.S. Cl. CPC A61B 17/1675; A61B 17/1615; A61B 17/1622; A61B 17/1631; A61B 17/1635

(57) ABSTRACT

Described herein are specialized methods and systems that may be utilized to secure a soft tissue graft to a boney surface. The methods and systems of the present invention facilitate the efficient and minimally invasive formation of sockets and/or tunnels in boney surfaces that may then serve as a site for graft placement by aperture and/or suspensory fixation means. The present invention has particular applicability to the surgical repair and reconstruction of torn or ruptured ligaments of the leg, such as anterior and posterior cruciate ligaments.

### PRIORITY

This application claims the benefit of U.S. Provisional Application Ser. No. 62/179,167 filed Apr. 29, 2015; 62/284,479 filed Oct. 1, 2015 and 62/284,714 filed Oct. 7, 2015, the contents of which are hereby incorporated by reference in their entirety.

### TECHNICAL FIELD OF THE INVENTION

The present invention relates generally to the field of arthroscopic surgery, more particularly, to methods and systems for securing a soft tissue graft to a boney surface. The invention has particular applicability to the surgical repair and reconstruction of torn or ruptured ligaments of the knee, such as the anterior and posterior cruciate ligaments.

### BACKGROUND OF THE INVENTION

Rupture of the anterior cruciate ligament (ACL) is a common injury in active people, and one of the most common knee injuries in sports. The healing response after ACL rupture is generally poor and, without surgical reconstruction, movement of the ACL deficient knee is limited. ACL reconstruction (repair) is one of the most common procedures performed by orthopedic surgeons and methods for performing the procedure are continually undergoing development. This development is partly fueled by the large number of athletes who require a rapid recovery in order to return to their sports in a timely manner. Since the early twentieth century, when ACL repair was first performed, there has been a constant evolution in techniques so as to reduce pain and speed the return to normal activities while improving post-surgical knee function.

In ACL and Posterior Cruciate Ligament (PCL) repair, a graft is affixed to the femur and tibia using cylindrical recesses (“tunnels”) formed in the respective bones. In ACL reconstruction, tunnels must be formed in the lateral femoral condyle, one of the two projections on the lower extremity of the femur, as well as in the femur itself. Fixation of the graft tissue to and in the tunnels may be accomplished using an interference screw implant, which is placed in the tunnel adjacent to the graft (a process referred to as “aperture fixation”), or by means of sutures affixed to the graft at one end and to an element external to the tibia or femur at the other (a process referred to as “suspensory fixation”). Illustrative examples of these elements include the GraftMax™ Button by Conmed, Inc. (Utica, N.Y.), Suture Buttons by Arthrex, Inc. (Naples, Fla.), and the Graft Fixation System (GFS) by Parcus Medical, Inc. (Sarasota, Fla.)

Historically, these tunnels were formed from the outside in, using guide pins and reamers. Recently, however, new “all inside” techniques have been developed that reduce trauma to the knee so as to reduce the patient's pain after surgery and reduce the recovery time. In these techniques, the tunnels into which the graft is to be affixed are formed from inside the knee. Some techniques use a special type of reamer that has a small diameter proximal shaft and a pivoting cutting element at the distal end. This device is inserted into the knee through a small diameter hole and, when inside the knee, the distal cutting element is pivoted so that a cutting element protrudes past the shaft diameter, with the proximal edge of the cutting element having a geometry for cutting bone to form a tunnel. After pivoting the cutting element, the drill is activated and the cutting device is proximally withdrawn so that the cutting element produces a larger diameter tunnel. When the desired tunnel length is achieved, the distal cutting element is retracted (by pivoting) and the device withdrawn through the smaller diameter hole proximal to the tunnel. An illustrative example of such a device is the FlipCutters® by Arthrex, Inc. (Naples, Fla.). While beneficial in theory, these “inside-out” reamer devices tend to fail during actual use. In one failure mode, the cutting element fractures during use so as to create a loose body within the joint, or more seriously, a fractured portion of the cutting element may be embedded in the bone. Additionally, the cutting element or the channel on the device into which the cutting element must be retracted for withdrawal from the femur may be damaged during use so as to prevent the cutting element from returning to its original position. In such cases, it becomes impossible to remove the device via the proximally located small diameter hole through which it was inserted. The surgeon must, in such cases, withdraw the device by cutting a tunnel of constant diameter to the lateral surface of the femur.

In another approach for forming femoral tunnels from the inside, a drill guide is inserted via the superomedial portal and located at the anatomical site for the tunnel. A flexible drill tip guide pin having a rigid distal portion is then inserted into the guide and a hole is drilled from the medial side of the lateral condyle to the lateral surface of the femur. The drill guide is then removed. A cannulated flexible reamer is thereafter passed over the flexible guide pin to the rigid distal portion and activated to form the femoral socket of the desired depth. Examples of such a system include the GraftMax™ Curved Reamer System by Conmed, Inc. (Utica, N.Y.). Certain elements of the GraftMax™ system are reusable and thus require cleaning and sterilization between cases. As such, they present opportunities for surgical site infection.

Similar to the Conmed GraftMax™ system, the VersiTomic® ACL Flexible Reaming System by Stryker, Inc. (Kalamazoo, Mich.) uses flexible reamers and a flexible guide pin. With the Stryker system, a flexible guide pin is placed from the inside of the knee via the superomedial portal and drilled through the lateral condyle to the lateral surface of the femur. Thereafter, a flexible reamer follows the flexible guide pin to produce the femoral tunnel. As with the GraftMax™ Curved Reamer System, certain components of the VersiTomic® reaming system are reusable, require cleaning and sterilization between cases and thus present opportunities for surgical site infection.

If aperture fixation of the graft within the femoral tunnel is used, a flexible driver is required for placement of the interference screw. If tapping is required to form a threaded socket for the screw, as in the case of a bone-tendon-bone (BTB) graft, a flexible tapping device is required. These flexible devices may allow the anchor or threading device to deviate from the desired path parallel to the axis of the socket unless a cannulated screw and guide wire are used.

A femoral tunnel may be formed in the lateral condyle using rigid linear guide pins and reamers from the anteromedial port if the knee is hyper-flexed to 120 degrees or more so that the linear devices are not blocked by the medial condyle. However, tunnels so produced frequently have an undesirably short length.

The formation of such tunnels according to prior art methods is accomplished using orthopedic power devices and systems. Typical of these are the Power 600 and Power 300 Systems by Arthrex, Inc. (Naples, Fla.), the System 7 Precision Power Tools by Stryker, Inc. (Kalamazoo, Mich.) and the PowerPro System by Conmed, Inc. (Utica, N.Y.). These power systems are predominantly electrically powered (either battery or from a console), though some pneumatic systems exist. The systems have a handpiece housing a drive motor, and a variety of attachments that may be removably affixed thereto. Examples of such attachments include, for example, various saws and rotary connectors for driving drills, reamers or guide pins. Devices placed in these rotary connectors tend to be either rigidly linear, or flexible.

Flexible drilling devices require a guiding means for use. In a first guiding method, a cannulated external drill guide with an angularly offset distal portion is used. The distal cutting portion of the drill exits the guide at the guide's distal end with a predetermined angular offset determined by the guide. Typical of such systems is the MicroFX™ microfracture system by Stryker, Inc. A drawback of this and other similar systems is that they are “two handed”. That is, the surgeon is required to hold the drill guide in position with one hand while controlling the drilling device with the other. It is therefore necessary for an assistant to position the camera.

An alternative guiding means is used in the Conmed GraftMax™ system and the VersiTomic® ACL Flexible Reaming System by Stryker, Inc. These systems use a flexible guide pin that is followed by a flexible cannulated reamer. The flexible guide pins have a rigidly linear distal portion that is placed using a powered drilling device and a drill guide, another two-handed operation. Thereafter, the proximal end of the flexible guide pin is inserted into the cannulation of a flexible cannulated reamer and the reamer advanced distally along the flexible portion of the guide pin. When the reamer distal cutting element reaches the rigid portion of the guide pin, the handpiece is activated and the tunnel formed to a desired depth. Thereafter the drill and guide pin are removed from the site. However, the use of a standard drilling system to produce holes that are angularly offset from the axis of the power device is problematic in that it requires a separate guiding means. Prior to drilling a hole, the guiding means must be placed to ensure the correct angular offset. Placing a flexible guide pin is a two handed operation. If an external drill guide is used, drilling the hole is a two-handed operation.

At the completion of the procedure, the handpiece and drilling attachment along with reusable guides and flexible reamers must be cleaned and sterilized. As noted previously, reuse of guides and flexible reamers may present opportunities for surgical site infections if not properly cleaned and sterilized after each use. In addition, the cleaning and sterilization of these devices between cases has an associated cost and may lead to scheduling difficulties.

Accordingly, there is a need in the surgical arts for simple reliable methods for forming femoral tunnels from the inside that use single-use disposable devices and allow anatomical positioning of the tunnel. Additionally, there is a need in the art for a method for forming an inside-out tibial tunnel that utilizes simple, single-hand, disposable, and/or single-use devices.

### SUMMARY OF THE INVENTION

A primary objective of the present invention is to provide a one-handed method of inside-out femoral fixation that allows a surgeon to drill a femoral tunnel in an anatomic location from the anteromedial portal using a single-use drilling device. In the context of the present invention, the drilling device preferably has a rigidly angularly offset distal portion that allows the surgeon to avoid the medial condyle and produce femoral tunnels of increased length as compared to rigid linear drilling devices. The drilling device is preferably powered by a standard arthroscopic shaver handpiece. In a preferred embodiment, the distal cutting element has a first portion that produces the tunnel diameter, and a second more distal portion having a reduced diameter like that of a machinists' center drill that minimizes “walking” of the drilling device distal end from the intended location as the surgeon begins drilling of the tunnel. In other embodiments, the reduced diameter distal portion is optionally absent. In still other embodiments, the distal cutting element is cannulated. Graduations and indicia may be optionally provided on the distal portion of the drilling device to indicate the distance from the distal end of the cutting element so that tunnels formed using the tunnel drilling device may be drilled to a predetermined depth as indicated by the graduations and indicia.

In certain embodiments, a second single-use drilling device, constructed like the tunnel-drilling device previously described but with a smaller diameter and elongate distal drilling element, is used in addition to the tunnel drilling device.

Arthroscopic shavers are used in virtually all arthroscopic procedures. Arthroscopic shaver and burrs tend to have a fixed (non-rotating) outer tubular member, and an inner rotating member, wherein each member has at its respective proximal end a hub that mounts to a shaver handpiece. The outer hub rigidly positions the outer tubular member relative to the handpiece while the inner hub transmits torque from the handpiece to the inner tubular member. An arthroscopy shaver handpiece is a source for rotational energy with a standardized interface through which a stationary outer member and rotating inner member may be removably mounted. Shavers and burrs for use with a shaver handpiece are produced at low cost and are predominantly single-use devices.

The present invention arises from the discovery that single-use drilling devices useful for endoscopic procedures may be constructed such that the rotational energy is supplied by a standard arthroscopy shaver handpiece. Furthermore, because the outer member is fixed to the handpiece and non-rotating, drilling devices in which the distal drilling element is angularly offset from the handpiece axis may be constructed, with the axis of the distal portion of the outer tubular member being angularly offset from the axis of the proximal portions and handpiece. The inner drive member of the device has a flexible, torque-transmitting portion in the region of the bend formed in the outer member. The angular offset may be established during manufacture of the device or may be formed by the surgeon at time of use to suit the procedure requirements. Endoscopic drilling devices of the present invention may be rigid linear devices, or may have a rigidly offset distal portion.

The drilling devices of the present invention differ from currently available pre-bent and conventional burrs in that the distal cutting element is constrained at the distal end of the outer tubular member by features which resist axial movement of the cutting element relative to the outer tubular member when axial forces are applied, as when drilling or when retracting a drill from a drilled hole. All are simple devices that may be produced at low cost and discarded after a single use. Associated advantages are that only a shaver handpiece is required to produce the femoral tunnel, with the added benefit that such a handpiece is already being used to prepare the site for replacement of the ligament. All of the drilling devices are single-use disposables. Thus, the time and cost of subsequent cleaning and sterilization of the drilling devices is eliminated along with the related opportunities for surgical site infections.

In a preferred embodiment, the method of the present invention is applied after the graft is harvested and the diameter for the femoral tunnel is determined, a tunnel drilling device of a proper size and with a suitable degree of angular offset is selected. As with prior art methods, remnants of the failed ACL are removed using a conventional arthroscopic shaver or radio frequency (RF) device. The anatomic location for the femoral tunnel on the lateral condyle in the intercondylar notch is identified and marked. For example, the step of marking of the location for the femoral tunnel may optionally include the step of forming a conical depression in the condyle surface using a surgical awl or similar impacting device, much like a machinist center-punching a metal surface to locate a starting point for a twist drill.

Once the size and location of the target tunnel is determined, a suitable tunnel drilling device is inserted into the knee via the anteromedial portal. The distal end cutting element of said tunnel drilling device is then positioned at the selected (identified and marked) anatomic location and used to drill a tunnel of a predetermined depth, after which the tunnel drilling device is removed from the site. Thereafter, a second drilling device is inserted into the tunnel via the anteromedial portal and a hole drilled from the distal end of the tunnel to the lateral surface of the femur. Then, using select methods and devices of the present invention, a tibial tunnel may be similarly formed and the graft may be positioned within the femoral tunnel and affixed therein using standard suspensory fixation methods and devices. The repair may then be completed with tensioning of the graft and fixation in the tibial tunnel.

In an alternate embodiment of the present invention involving suspensory femoral fixation of a graft, after the femoral tunnel is formed using the tunnel drilling device as previously described, instead of using the second drilling device, a drill guide apparatus, commonly referred to as an “aimer”, may be used to define a path from a selected site on the lateral side of the femur to the lateral end of the tunnel previously formed. A drill tip guide wire or other drill may then be used to form a passage from the lateral surface of the femur to the lateral end of the tunnel. In a preferred embodiment, a drill tip guide pin is constructed as previously herein described so that it may be driven by a shaver handpiece. In other embodiments, the guide pin may be of conventional construction and may be driven by a conventional prior art device.

In yet another embodiment, the smaller diameter second drilling device may be used to create a small diameter pilot hole at the anatomic location for the femoral tunnel for the tunnel drilling device to follow initially. In still another embodiment, a drill tip guide pin may be placed from the outside using an “aimer”, a method commonly used for locating a femoral tunnel. The distal end of the guide pin then exits the lateral condyle at the anatomical location for the tunnel. Thereafter, a tunnel drilling device as previously herein described—i.e., a device having a cannulated distal end cutting element with a cannulation sized to fit over the distal end of the guide pin—may be introduced via the anteromedial portal to the site for the femoral tunnel. The cannulation of the drilling element slidably and rotatably engages the guide pin and the tunnel may then be drilled to the desired depth. Placement of the drill tip guide pin may be accomplished using a conventional powered handpiece or may utilize a shaver handpiece in accordance with the principles of the present invention.

In embodiments previously herein described, a passage is formed from the lateral end of the femoral tunnel to the lateral surface of the femur such that sutures affixed to the graft may be used to pull the graft into position and secure it in the tunnel by suspensory fixation means. However, the present invention is not so limited and thus finds utility in connection with alternate fixation methods, for example, femoral aperture fixation using an interference screw, wherein a passage for sutures from the tunnel to the femur lateral surface is not required. According to the principles of the present invention, an interference screw may be placed using a cannulated implant placement system in which sutures may be drawn through the system to the proximal end of a handle portion and cleated thereto to maintain tension on the sutures. Such an implant placement system preferably has a non-rotating distal portion that protrudes beyond the interference screw. Moreover, the distal portion of the implant placement system is preferably angularly offset from the proximal portion, the degree of the offset being equal to that of the distal portion of the drilling devices used to form the tunnel as previously herein described. Such implant placement systems and their use are described in detail in U.S. Pat. No. 9,226,817, as well as in co-pending U.S. application Ser. No. 15/012,060 filed Feb. 1, 2016, the respective contents of which are hereby incorporated herein by reference in their entirety.

When the materials and methods of the present invention are applied in the context of interference screw (aperture) fixation, the steps for forming the femoral tunnel are the same as previously herein described, with the exception that drilling of the passage from the tunnel to the femur lateral surface is eliminated. However, the anatomic location for the tunnel is identified and marked by methods previously described and the tunnel is formed using the drilling devices with an angular offset to their distal portions as previously herein described. The graft is prepared using conventional methods, with distal and proximal sutures attached. In the case of bone-tendon-bone (BTB) grafts, holes are drilled in the bone plugs for passage therethrough of sutures.

In a preferred embodiment, such holes may be drilled using a drilling device of the present invention powered by a shaver handpiece. In other embodiments, conventional drilling devices may be used. Thereafter the distal sutures may be drawn into the cannulation of an anchor placement system. The graft may then be releasably secured to the non-rotating distal portion of the system that protrudes beyond the interference screw by pulling on the portion of the sutures passing from the proximal handle portion, tensioning them, and then removably securing the sutures to the handle portion using cleats formed therein. Subsequently, the distal portion of the implant placement system with the end of graft secured thereto may be inserted into the femoral tunnel to the desired depth. The interference screw may then be threaded into the tunnel so as to secure the graft in the conventional manner. The sutures may then be uncleated and the placement system may be removed from the site. Thereafter, the sutures may be trimmed so as to complete the femoral fixation portion of the ACL replacement. Subsequent steps of the repair may be completed using standard methods.

In certain instances, such as when using aperture fixation for a bone-tendon-bone (BTB) graft, it may be necessary to form a threaded socket in which the interference screw is subsequently placed. These threads may be formed using an endoscopic tapping device having a distal portion that is angularly offset from the proximal portions, with the degree of the angular offset being equal to that of the drilling device(s) and the implant placement system. An endoscopic device of the type suitable for use in connection with surgical methods of the present invention is described in detail in U.S. Pat. No. 9,226,817, the entire contents of which are incorporated herein by reference. After the BTB graft is positioned within the femoral tunnel, the distal threading element of the tapping device is threaded into the juncture between the bone plug and the tunnel wall so as to form a threaded socket. Following this, the interference screw is placed as previously described.

Unlike prior art methods for forming a femoral tunnel for ACL reconstruction, the methods of the present invention allow the surgeon to form a femoral tunnel in an anatomic position from the inside of the knee, with the tunnel having increased length compared to those which may be produced from the inside using prior art rigid linear devices. Prior art methods for producing a femoral tunnel in an anatomic location from the inside using flexible guide pins and flexible reamers require the surgeon to perform added steps that add to procedure time and thus increase the risk of downstream problems. Additionally, the devices used to produce a femoral tunnel using methods of the present invention are low cost and single-use and can be powered by a standard arthroscopy shaver handpiece.

The formation of a tibial tunnel constitutes another aspect of the present invention. The methods associated with tibial tunnel formation are generally analogous to those used to form a femoral tunnel in that single-use devices powered by a shaver handpiece are used. However, unlike the devices used to form the femoral tunnel, those for forming the tibial tunnel are rigid linear devices. For example, a prior art drill guide such as is conventional in the art, a drill tip guide pin of the present invention may be placed in position. Next, the proximal portion of the guide pin with the hub assembly attached thereto may be broken off so that the drill guide can be removed. Thereafter, a cannulated drilling device of the diameter required for forming the tunnel may be selected and loaded into the shaver handpiece. The tunnel may then be drilled with the previously placed guide pin providing the positioning. After use, the drill tip guide pin and cannulated drilling device may be discarded.

These and other aspects are accomplished in the invention herein described. Further objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention. In particular, while the invention is described herein with reference to a number of specific embodiments, it will be appreciated that the description is illustrative of the invention and is not constructed as limiting of the invention. For example, although the present invention has been previously herein described with reference to ACL reconstruction for simplicity only, it is readily apparent that the same methods may be used for reconstruction of the PCL with references to lateral and medial structures and directions modified as appropriate.

### BRIEF DESCRIPTION OF THE FIGURES

Various aspects and applications of the present invention will become apparent to the skilled artisan upon consideration of the brief description of figures and the detailed description of the present invention and its preferred embodiments that follows:

FIG. 1 is a perspective view of a first endoscopic drilling device for an ACL/PCL repair method of the present invention.

FIG. 2 is a side elevational view of the objects of FIG. 1.

FIG. 3 is an expanded view of the objects of FIG. 2 at location B.

FIG. 4 is a perspective view of a second endoscopic drilling device configured for forming a tunnel and an element of an ACL repair system of the present invention.

FIG. 5 is a side elevational view of the objects of FIG. 4.

FIG. 6 is an expanded view of the objects of FIG. 5 at location A.

FIG. 7 is a perspective view of an endoscopic tapping device for an ACL repair system of the present invention.

FIG. 8 is a side elevational view of the objects of FIG. 7.

FIG. 9 is an expanded view of the objects of FIG. 8 at location A.

FIG. 10 is a perspective view of an endoscopic implant placement system for an ACL repair system of the present invention.

FIG. 11 is a side elevational view of the objects of FIG. 10.

FIG. 12 is an expanded view of the objects of FIG. 11 at location A.

FIG. 13 depicts a first endoscopic drilling device positioned within a knee in preparation for the first step of an ACL repair method of the present invention.

FIG. 14 depicts a knee after completion of the first step.

FIG. 15 depicts a second endoscopic drilling device drilling a femoral tunnel in a second step of the repair method of the present invention.

FIG. 16 depicts a knee after the completion of the second step with the femoral tunnel formed.

FIG. 17 depicts a first endoscopic drilling device forming a hole between the distal end of the femoral tunnel of FIG. 15 and the back surface of the femur in a third step of the ACL repair method of the present invention.

FIG. 18 depicts the knee at the completion of the third step.

FIG. 19 depicts the knee wherein a tibial tunnel has been formed in the fourth step of the present method.

FIG. 20 depicts a soft tissue graft positioned in preparation for drawing into the knee for femoral fixation, the fifth step of the present method.

FIG. 21 depicts the graft of FIG. 20 positioned within the knee in preparation for femoral fixation.

FIG. 22 depicts the graft of FIG. 20 positioned within the knee and the femoral portion thereof fixed within the femur using a button, the sixth and final step in the femoral side fixation portion of the ACL repair method of the present invention.

FIG. 23 is a plan view of a drill guide of the present invention.

FIG. 24 is a perspective view of the drill guide of FIG. 23.

FIG. 25 depicts an alternate method for forming the hole of the third step of the present method using the drill guide of FIG. 23, the guide being positioned in preparation for drilling the hole.

FIG. 26 depicts the knee after completion of the hole, the knee condition corresponding to that depicted in FIG. 19.

FIG. 27 is a perspective view of the distal end of a third cannulated drilling device for an alternate embodiment ACL repair system and method of the present invention.

FIG. 28 is a side elevational view of the objects of FIG. 27.

FIG. 29 depicts a knee in which a guide wire is placed according to the principles of prior art ACL repair procedures.

FIG. 30 depicts the knee of FIG. 29 with the third cannulated drilling device of FIG. 27 positioned for drilling a femoral tunnel in accordance with the principles of an alternate embodiment of the present invention.

FIG. 31 depicts the knee and drilling device of FIG. 30 with the drilling device forming the femoral tunnel.

FIG. 32 depicts the knee at the completion of tunnel formation, the knee condition corresponding to that depicted in FIGS. 19 and 26.

FIG. 33 depicts a knee with second drilling device of FIG. 3 positioned for forming a femoral tunnel in an alternate embodiment of the ACL repair method of the present invention.

FIG. 34 depicts the knee and drilling device of FIG. 33 with the drilling device forming the femoral tunnel.

FIG. 35 depicts the knee with the tunnel formed.

FIG. 36 depicts the knee of FIG. 35 with the tibial tunnel formed.

FIG. 37 depicts the knee of FIG. 36 with a soft tissue graft inserted into the knee through the tibial tunnel with the distal sutures loaded into the implant placement system of FIG. 10.

FIG. 38 depicts the elements of FIG. 37 with the sutures tensioned and cleated so as to draw the distal end of the graft to the distal end of the implant placement system.

FIG. 39 depicts the elements of FIG. 37 with the distal end of the implant system and the distal end of the graft releasably attached thereto inserted in the femoral socket.

FIG. 40 depicts the elements of FIG. 39 with the implant placement system in use threading an implant into the socket so as to secure the distal end of the graft.

FIG. 41 depicts the knee with the distal end of the graft secured in the socket by a threaded implant.

FIG. 42 depicts a prior art bone-tendon-bone (BTB) graft prepared with sutures for use in an ACL repair.

FIG. 43 depicts the BTB graft of FIG. 42 positioned within a knee as in an ACL repair with femoral button fixation, corresponding to the soft tissue graft repair depicted in FIG. 22 and accomplished by the related methods previously herein illustrated.

FIG. 44 depicts a BTB graft positioned within a knee in preparation for femoral fixation thereof by an alternate method of the present invention.

FIG. 45 depicts the knee and graft of FIG. 44 with the endoscopic tapping device of FIG. 6 positioned in the knee in preparation for forming threads in the femoral socket and the bone portion positioned therein.

FIG. 46 depicts the knee and BTB graft of FIG. 45 with the threading step completed.

FIG. 47 depicts the elements of FIG. 46 further, wherein the implant system of FIG. 10 positioned in preparation for placing an interference screw implant to secure the distal bone portion of the graft in the femoral tunnel.

FIG. 48 depicts the knee and BTB graft with femoral fixation by the interference screw implant of FIG. 47, and the distal sutures untrimmed.

FIG. 49 depicts the elements of FIG. 48 wherein supplemental button fixation has been added and the distal sutures trimmed.

FIG. 50 is a side elevational view of a drill tip guide pin of the present invention mounted in an arthroscopy shaver handpiece.

FIG. 51 is an expanded view of the objects of FIG. 50 at location B.

FIG. 52 is a perspective view of a twist drill of the present invention mounted in an arthroscopy shaver handpiece.

FIG. 53 is a side elevational view of the objects of FIG. 52.

FIG. 54 is a side elevational view of a cannulated reaming device of the present invention mounted in a shaver handpiece.

FIG. 55 is an expanded sectional view of the objects of FIG. 54 at location A-A.

FIG. 56 depicts drill tip guide pin of FIG. 50 mounted in a shaver handpiece placed in preparation for forming of a tibial tunnel using a prior art drill guide.

FIG. 57 depicts the elements of FIG. 56 but with the shaver handpiece and proximal guide pin portion removed in a next step of tunnel placement.

FIG. 58 depicts the guide pin in position with the drill guide removed.

FIG. 59 depicts a cannulated reamer of the present invention positioned over the guide pin previously placed in preparation for forming a tibial tunnel.

FIG. 60 depicts the elements of FIG. 59 with the cannulated reamer in position at the completion of forming the tibial tunnel.

FIG. 61 depicts the knee at the completion of the femoral and tibial tunnels.

### DETAILED DESCRIPTION OF THE PREFERRED EMBODIMENTS

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims. Accordingly, unless otherwise defined, all technical and scientific terms used herein have the same meaning as commonly understood by one of ordinary skill in the art to which the present invention belongs. However, in case of conflict, the present specification, including definitions below, will control.

In the context of the present invention, the following definitions apply:

The words “a”, “an” and “the” as used herein mean “at least one” unless otherwise specifically indicated. Thus, for example, reference to an “opening” is a reference to one or more openings and equivalents thereof known to those skilled in the art, and so forth.

The term “proximal” as used herein refers to that end or portion which is situated closest to the user of the device, farthest away from the target surgical site. In the context of the present invention, the proximal end of an arthroscopic device of the present invention includes the driver and handle portions.

The term “distal” as used herein refers to that end or portion situated farthest away from the user of the device, closest to the target surgical site. In the context of the present invention, the distal end of an arthroscopic repair system of the present invention includes one or more components adapted to address the patient's body, for example, a distal drilling element.

In the context of the present invention, the term “cannula” is used to generically refer to the family of rigid or flexible, typically elongate lumened surgical instruments that facilitate access across tissue to an internally located surgery site.

In the context of the present invention, the term “cannulated” is used to generically refer to the family of rigid or flexible, typically elongate surgical instruments having a central lumen into which an elongate device such as a guide wire or guide pin may pass so as to prevent the cannulated device from deviating from a selected path during use.

The terms “tube” and “tubular” are interchangeably used herein to refer to a generally round, long, hollow component having at least one central opening often referred to as a “lumen”.

The terms “lengthwise” and “axial” are used interchangeably herein to refer to the direction relating to or parallel with the longitudinal axis of a device. The term “transverse” as used herein refers to the direction lying or extending across or perpendicular to the longitudinal axis of a device.

The term “lateral” pertains to the side and, as used herein, refers to motion, movement, or materials that are situated at, proceeding from, or directed to a side of a device or patient.

The term “medial” pertains to the middle, and as used herein, refers to motion, movement or materials that are situated in the middle, in particular situated near the median plane or the midline of the device or subset component thereof or of a patient.

The present invention finds particular utility in connection with arthroscopic repair and reconstruction of the ligaments of knee, particularly anterior cruciate ligament (ACL) and the posterior cruciate ligament (PCL). The ligaments in the knee connect the femur (thighbone) to the tibia (shin bone), and include the following:
the anterior cruciate ligament (ACL);
the posterior cruciate ligament (PCL);
the medial collateral ligament (MCL); and
the lateral collateral ligament (LCL).

The ACL is one of a pair of cruciate ligaments (the other being the posterior cruciate ligament), also called cruciform ligaments as they are arranged in a crossed formation. The ACL provides 85% of the restraining force to anterior tibial displacement at 30 degrees and 90 degrees of knee flexion. The ACL originates from deep within the notch of the distal femur. Its proximal fibers fan out along the medial wall of the lateral femoral condyle, one of the two projections on the lower extremity of the femur, the other being the medial condyle. There are two bundles of the ACL—the anteromedial (located in the front and toward the middle) and the posterolateral (located behind and to one side, specifically to the outer side), named according to where the bundles insert into the tibial plateau, a critical weight-bearing region on the upper extremity of the tibia. The ACL attaches in front of the intercondyloid eminence of the tibia (a region composed of the medial and lateral intercondylar tubercle that divides the intercondylar area into an anterior and posterior area), being blended with the anterior horn of the medial meniscus.

The PCL connects the posterior intercondylar area of the tibia to the medial condyle of the femur and gets its name by attaching to the posterior portion of the tibia. This configuration allows the PCL to resist forces pushing the tibia posteriorly relative to the femur. The PCL is located within the knee joint where it stabilizes the articulating bones, particularly the femur and the tibia, during movement. It originates from the lateral edge of the medial femoral condyle and the roof of the intercondyle notch then stretches, at a posterior and lateral angle, toward the posterior of the tibia just below its articular surface.

Both the ACL and the PCL are designated as an “intracapsular ligaments” because they lie deep within the knee joint. Both are isolated from the fluid-filled synovial cavity, with the synovial membrane wrapped around them.

As discussed above, when a tissue, more particularly a soft connective tissue in a joint space, becomes damaged or torn from its associated bone or cartilage, surgery is usually required to reattach the tissue or reconstruct the bone. The present invention is directed to select means and mechanisms for securing a torn, damaged or displaced tissue, such as a ligament or a tendon, to the boney tissue associated therewith, such as the femur, knee or tibia.

As used herein, the term “tissue” refers to biological tissues, generally defined as a collection of interconnected cells that perform a similar function within an organism. Four basic types of tissue are found in the bodies of all animals, including the human body and lower multicellular organisms such as insects, including epithelium, connective tissue, muscle tissue, and nervous tissue. These tissues make up all the organs, structures and other body contents. While the present invention is not restricted to any particular soft tissue, aspects of the present invention find particular utility in the repair of connective tissues such as ligaments or tendons, particularly those of the knee joint.

In a similar fashion, while the present invention is not restricted to any particular boney tissue, a term used herein to refer to both bones and cartilage, aspects of the present invention find particular utility in the repair or reattachment of connective tissues to the boney elements of the leg.

When the damaged tissue is of sufficient quantity and quality, the damaged portion may simply be directly reattached to the bone from which it was torn so that healing back to the bone can take place. However, in other situations, a “graft” may be needed to stimulate regrowth and permanent attachment. In the context of the present invention, the term “graft” refers to any biological or artificial tissue being attached to the boney tissue of interest, including:
Autografts, i.e., grafts taken from one part of the body of an individual and transplanted onto another site in the same individual, e.g., ligament graft;
Isografts, i.e., grafts taken from one individual and placed on another individual of the same genetic constitution, e.g., grafts between identical twins;
Allografts, i.e., grafts taken from one individual placed on genetically non-identical member of the same species; and
Xenografs, i.e., grafts taken from one individual placed on an individual belonging to another species, e.g., animal to man.
   Autografts and isografts are usually not considered as foreign and, therefore, do not elicit rejection. Allografts and xenografts are recognized as foreign by the recipient thus carry a high risk of rejection. For this reason, autographs and isografts are most preferred in the context of the present invention.

Surgical interventions such as contemplated herein generally require the boney tissue to be prepared for receiving the graft. In the context of the present invention, such preparation includes the formation of a “socket” or “tunnel”, i.e., a hole punched or drilled into the bone into which a graft-associated fixation mechanism, such as an interference screw or suture implant, may be received. In the context of the present invention, the terms “socket” and “tunnel” may be used interchangeably or, alternatively, the term “socket” may be used to refer to a single, preferably interior-opening hole or hollow whereas the term “tunnel” may be used herein to refer to a “through-and-through” passage having both interior and exterior openings. The socket or tunnel may be prepared at the desired target location using conventional instruments such as drills, taps, punches or equivalent hole-producing devices. In the context of the present invention, the femoral “tunnel” is preferably formed from the “inside out” rather than the “outside in”.

While certain procedures contemplate directly attaching the graft to the bone, the more common route involves the employment of an implant specially configured to hold and/or enable attachment of the graft to the boney tissue. As used herein, the term “implant” refers to a prosthetic device fabricated from a biocompatible and/or inert material. In the context of the present invention, examples of such “implants” include conventional and knotless anchors of both the screw-threaded and interference-fit variety.

The present invention makes reference to insertion devices commonly referred to in the art as “drills” and “drivers”, i.e., devices that “drill” the tunnel and “drive” the graft and/or fixation device into the tunnel. In the context of the present invention, the drills and drivers may be conventional, e.g., rigidly linear as previously herein described, such as for use in the context of a tibal tunnel, or, as discussed in detail herein, “off-axis”, e.g., having an angularly offset distal portion adapted to drill off-axis femoral tunnels such as described in detail above.

In the context of the present invention, reference is made to various lock-and-key type mating mechanisms that serve to establish and secure the axial and rotational arrangement of various concentric or relatively slidable device components. It will be readily understood by the skilled artisan that the position of the respective coordinating elements (e.g., recessed slots and grooves that mate with assorted projecting protrusions, protuberances, tabs and splines) may be exchanged and/or reversed as needed.

In certain embodiments, the present invention contemplates securing a graft to an tunnel or a tunnel implant via sutures. In the context of the present invention, the term “suture” refers to a thread-like strand or fiber used to hold body tissues after surgery. Sutures of different shapes, sizes, and thread materials are known in the art and the present invention is not restricted to any particular suture type. Accordingly, in the context of the present invention, the suture may be natural or synthetic, monofilament or multifilament, braided or woven, permanent or resorbable, without departing from the spirit of the invention.

The instant invention has both human medical and veterinary applications. Accordingly, the terms “subject” and “patient” are used interchangeably herein to refer to the person or animal being treated or examined. Exemplary animals include house pets, farm animals, and zoo animals. In a preferred embodiment, the subject is a mammal, more preferably a human.

Hereinafter, the present invention is described in more detail by reference to the Figures and Examples. However, the following materials, methods, figures, and examples only illustrate aspects of the invention and are in no way intended to limit the scope of the present invention. For example, while the present invention makes specific reference to arthroscopic procedures, it is readily apparent that the teachings of the present invention may be applied to other minimally invasive procedures and are not limited to arthroscopic uses alone. As such, methods and materials similar or equivalent to those described herein can be used in the practice or testing of the present invention.

### Examples

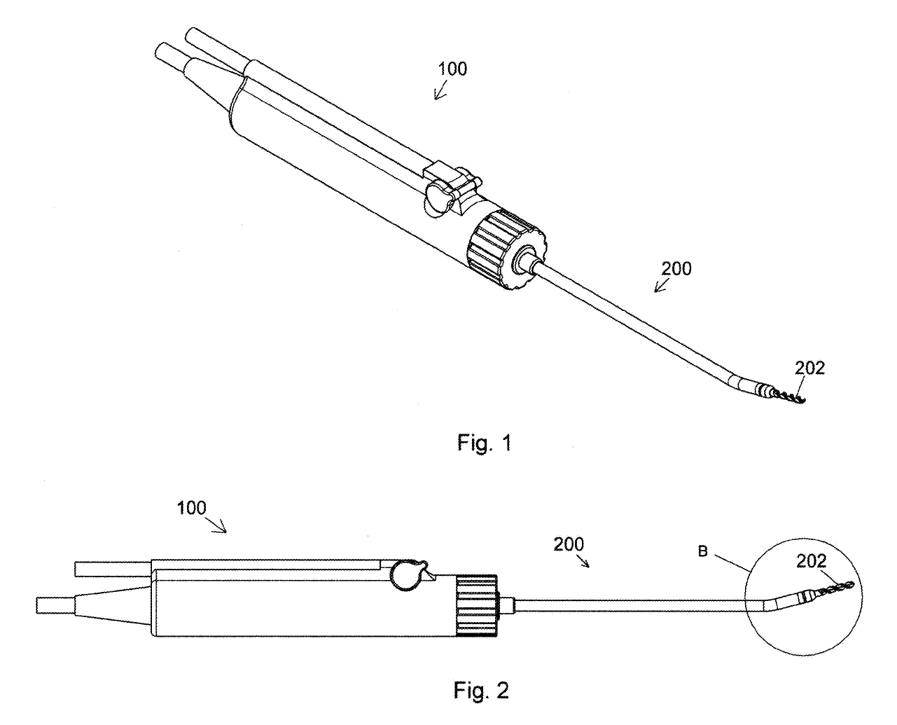

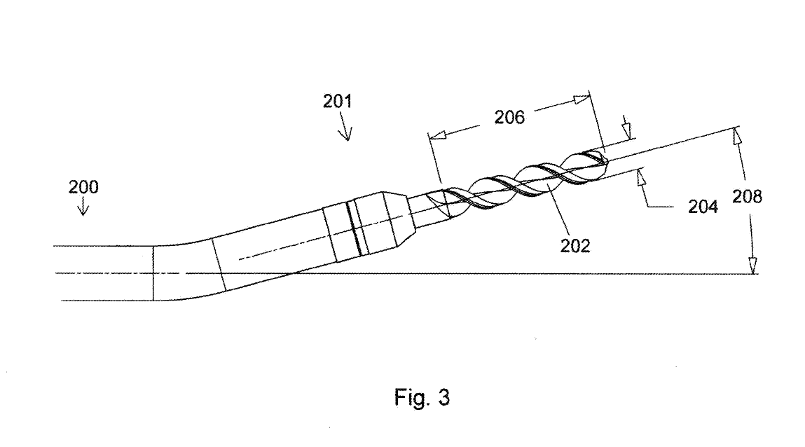

FIGS. 1 through 3 depict a first endoscopic drilling device 200 powered by an endoscopic shaver handpiece 100. Distal portion 201 is angularly offset from the axis of the shaver hand piece 100 angle 208 that ranges from 2 to 40 degrees, preferably 3 to 30 degrees, and more preferably 5 to 25 degrees. Distal cutting (drilling) element 202 has a diameter 204 preferably between one and five millimeters and more preferably between 1.5 and 4 millimeters. Cutting element 202 has an effective length 206 preferably between three and thirty millimeters, and more preferably between ten and twenty-five millimeters.

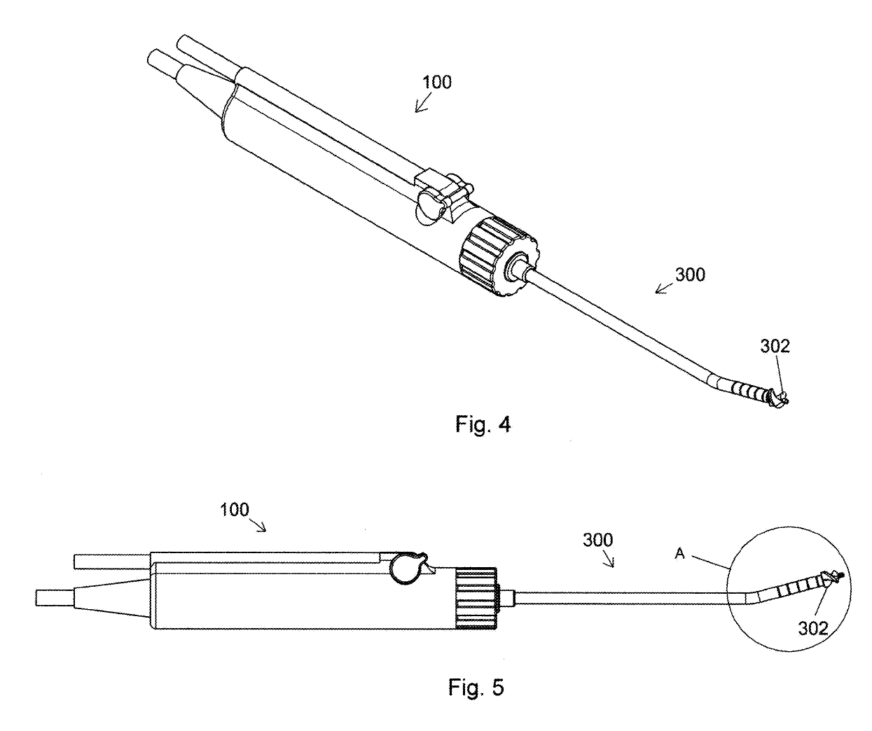

Referring now to FIGS. 4 through 6, endoscopic tunnel drilling device 300 is powered by endoscopic shaver hand piece 100. Distal portion 301 is angularly offset from the axis of the shaver handpiece 100 angle 308, which is equal to angle 208 of first drilling device 200. Distal cutting element 302 has a diameter 304 preferably between 4 and 16 millimeters and more preferably between 5 and 13 millimeters. Distal portion 303 of cutting element 302 has a reduced diameter. Distal portion 301 of drilling device 300 has formed thereon depth markings 309 and associated indicia 307 so as to indicate the depth of a hole formed using drilling device 300.

In a preferred embodiment, drilling devices 200 and 300 are discarded after a single use.

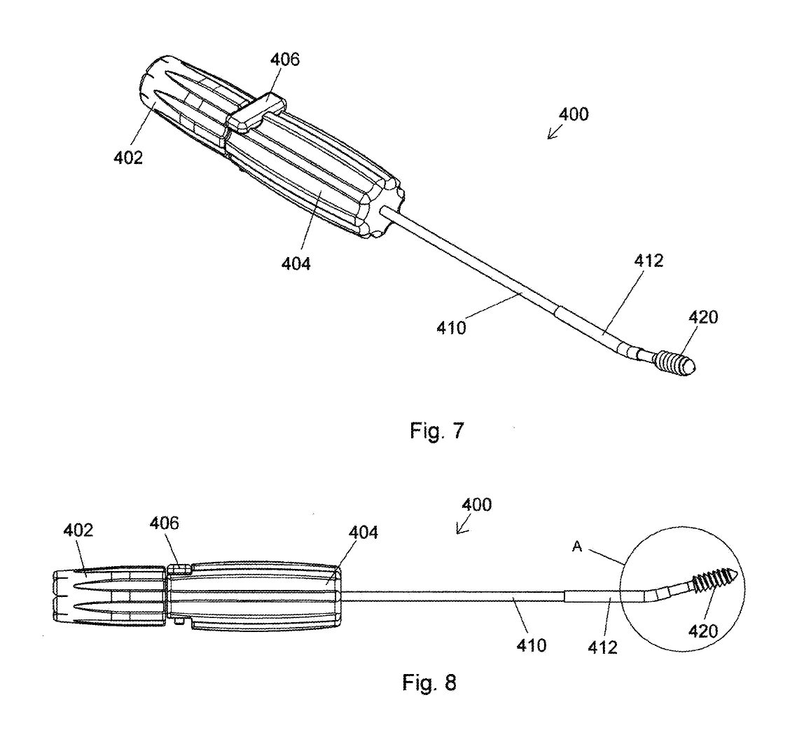

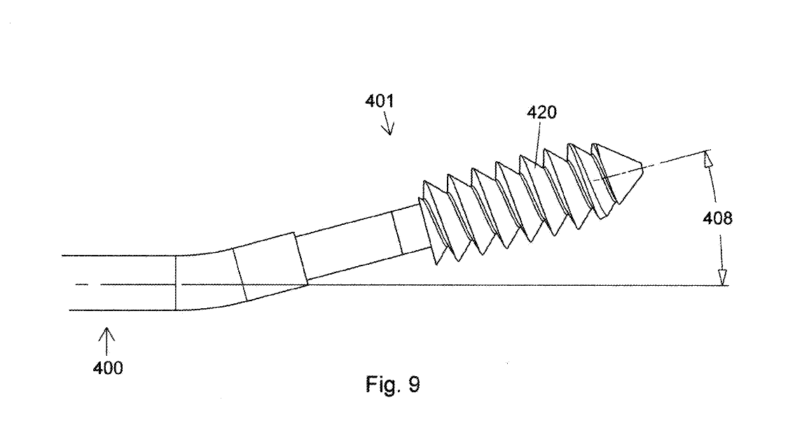

An endoscopic tapping device 400 is depicted in FIGS. 7 through 9 and described in detail in U.S. Pat. No. 9,226,817 referenced above, the contents of which are incorporated by reference herein. Tapping device 400 has a non-rotating proximal handle 402 and a rotating distal handle 404 which through elongate element 410 and flexible drive element 412 provides torque to thread forming element 420. Distal portion 401 of tapping device 400 is angularly offset 408 from elongate element 410, offset 408 being equal to angular offsets 208 and 308 of first drilling device 200 and second drilling device 300 respectively. Proximal handle 402 and distal handle 404 are rotatably connected by key 406

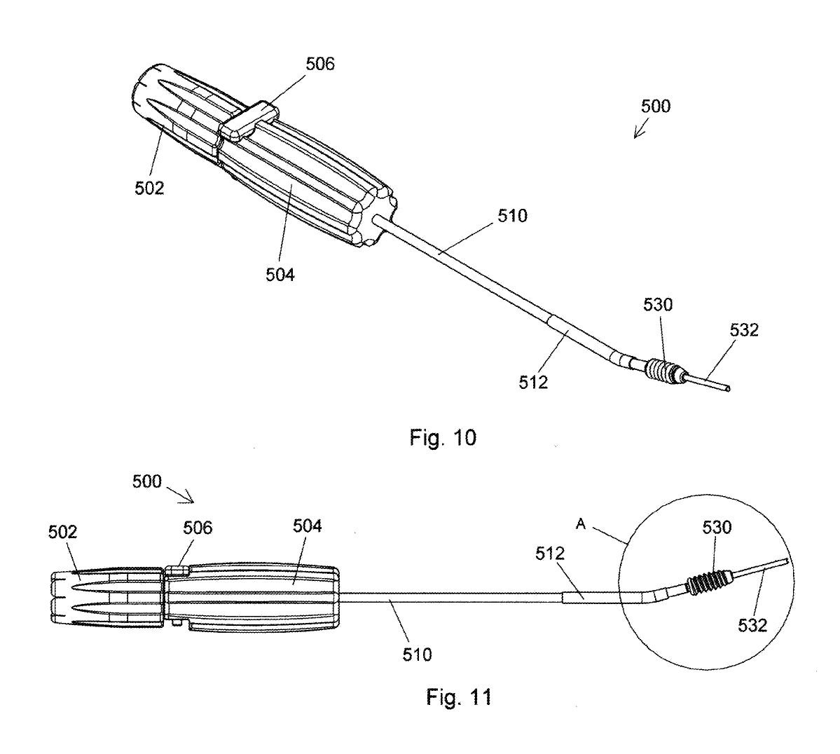

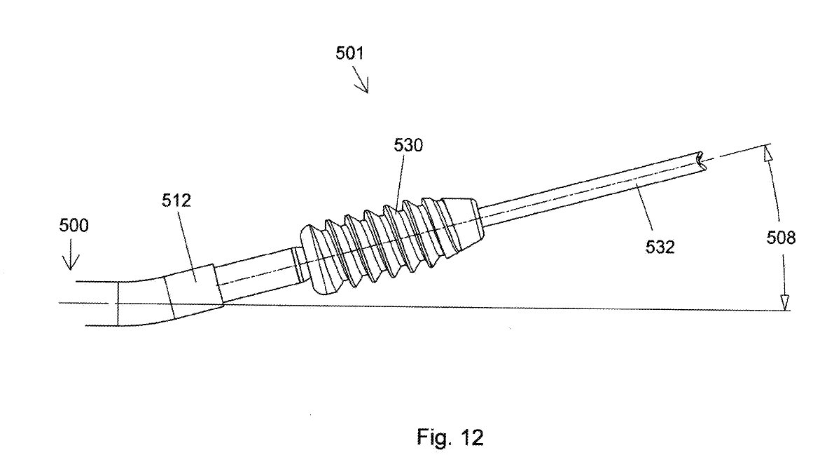

FIGS. 10 through 12 depict an implant placement system 500 (described in detail in U.S. Pat. No. 9,226,817) that has a non-rotating proximal tensioning handle 502 and a rotatable, axially translatable driver handle 504 which through elongate element 510 and flexible drive element 512 provides torque to implant 530, implant 530 being a threaded interference screw. Tubular non-rotating distal tensioning element 532 protrudes distally beyond interference screw 530. Key 506 prevents relative rotation and axial movement between non-rotating proximal handle 502 with its associated tensioning elements, and distal handle 504 with its torque transmitting elements. Removing key 506 allows distal handle 504 to rotate and advance distally so as to bring implant 530 to a prepared tunnel and to thread it therein.

Hereafter are described methods for performing ligament reconstruction, more particularly an ACL repair commensurate with the present invention. To that end, methods of the present invention allow for the formation of a femoral tunnel using endoscopic drilling devices having a distal portion that is rigidly angularly offset from the proximal portion. This allows the device to avoid the medial condyle such that an anatomic femoral socket may be formed without a notchplasty (removal of bone in the intercondylar notch) or possible damage to the medial condyle. In methods of the present invention, either or both of suspensory (button) fixation and aperture (interference screw) fixation may be used, alone or in combination. If aperture fixation is used, the interference screw may be placed according to principles of the present invention, using a system in which the distal portion of the placement system is rigidly angularly offset from the proximal portion so that the implant may be placed with the implant axis parallel to or coaxial to the tunnel axis. Ligament repair methods of the present invention allow direct anatomic placement of the femoral tunnel with access from the anteromedial portal without requiring hyper-flexing of the knee because the angular offset of the devices used allows access while bypassing the medial condyle. Tunnels formed using methods and devices of the present invention may have a greater length than those formed by standard rigid linear devices using anteromedial portal access and hyper flexing of the knee.

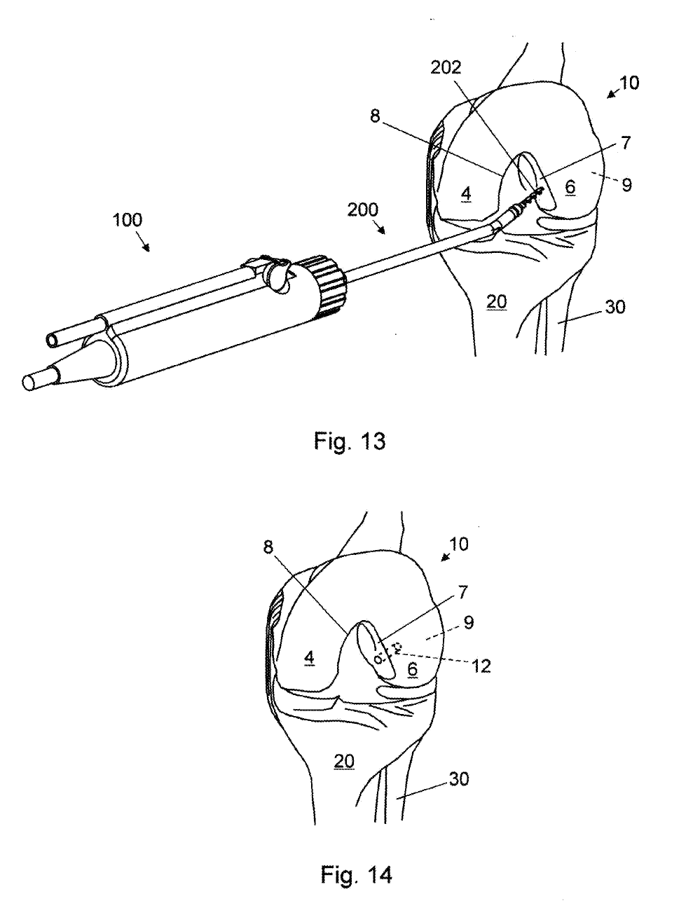

FIG. 13 depicts a knee having a femur 10, tibia 20 and fibula 30. Femur 10 has a medial condyle 4 and lateral condyle 6 separated by intercondylar notch 8. First endoscopic drilling device 200 mounted in shaver handpiece 100 is inserted into the knee via the anteromedial portal. The distal end of drilling device 200, including cutting element 202, is positioned at the anatomical location for the tunnel on medial surface 7 of the lateral condyle 8, the angular offset of the distal portion of drilling device 200 allowing the drill to “reach around” medial condyle 4 to access locations which cannot be accessed with a rigidly linear device. Handpiece 100 is activated and an initial locating hole 12 is formed in femur lateral condyle 6 as depicted in FIG. 14. Drilling device 200 may then be removed from the handpiece 100 and set aside or discarded, as needed.

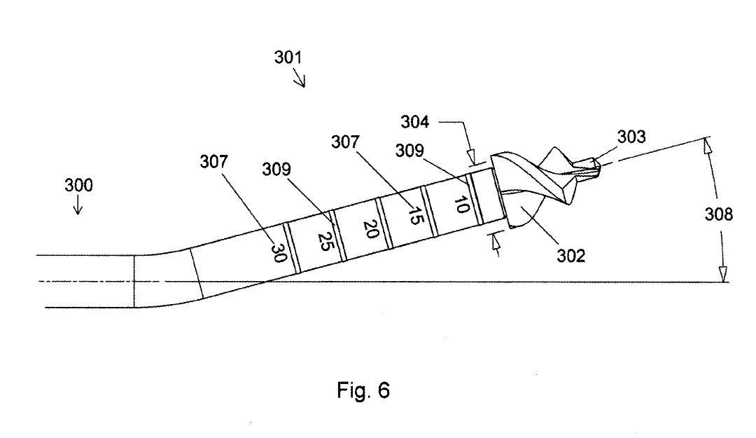

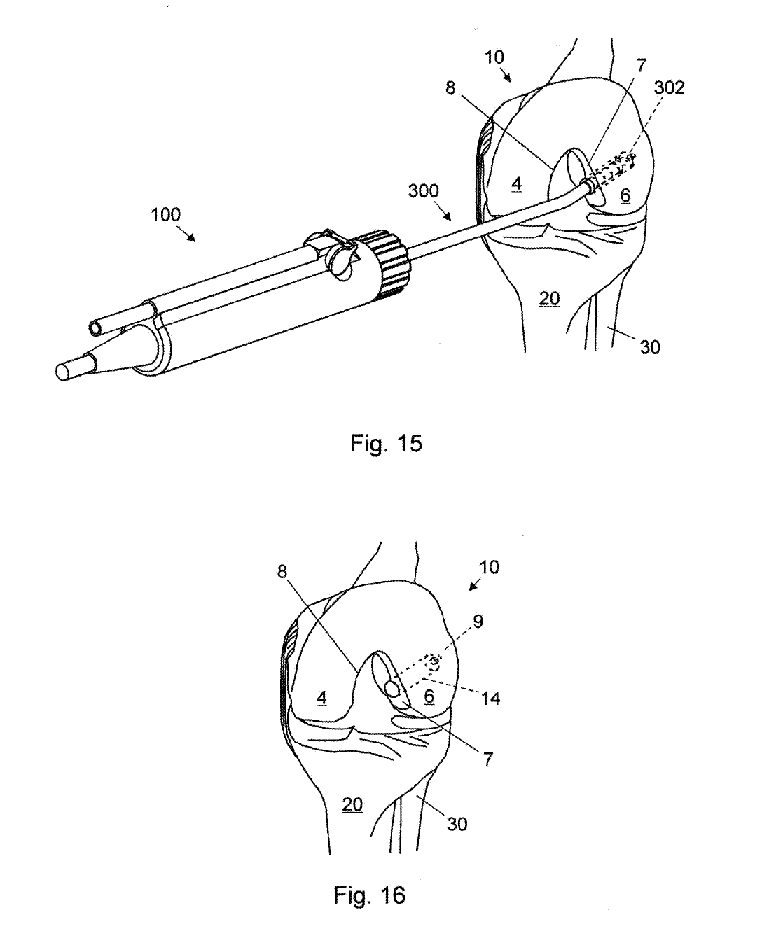

The desired tunnel diameter is determined based on the cross-section of the graft to be introduced. Next, a tunnel drilling device 300 of appropriate diameter is selected and mounted in shaver handpiece 100 and positioned in the knee in the same manner as used for drilling device 200 previously. Handpiece 100 is activated and a tunnel formed by distal end cutting element 302 as depicted in FIG. 15. Drilling device 300 is advanced to the desired tunnel length indicated by depth marking 309 and indicia 307 (FIG. 6). FIG. 16 depicts tunnel 14 formed in lateral condyle 6.

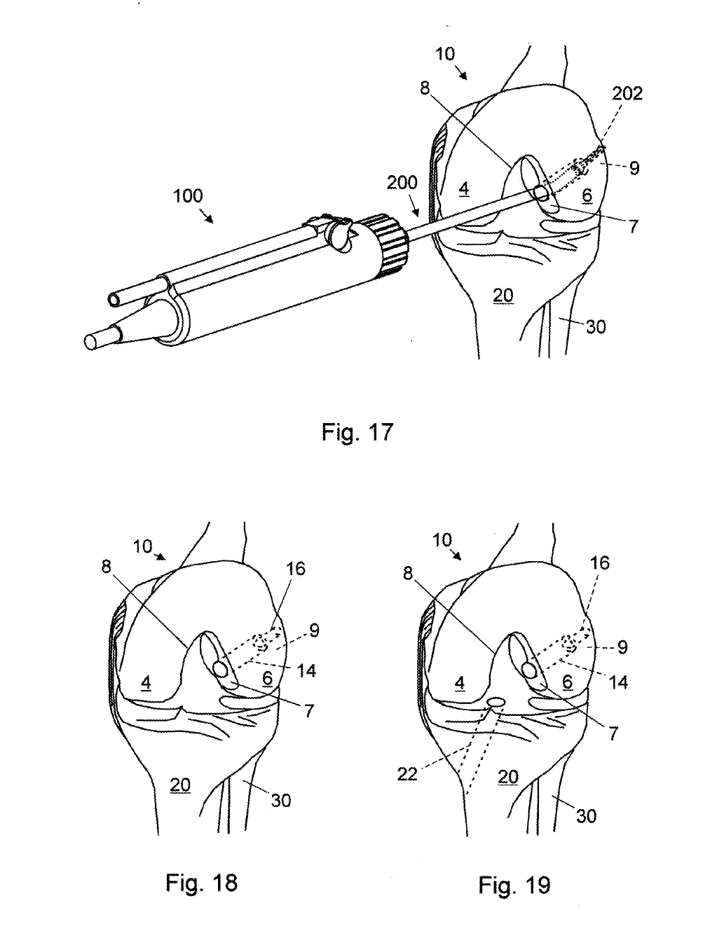

In FIG. 17, first drilling device 200 is positioned within socket 14 in preparation for forming a hole 16 to lateral surface 9 of femur 10 from the distal end of socket 14 as depicted in FIG. 18. Thereafter, the tibial tunnel 22 is formed using standard methods and devices as depicted in FIG. 19.

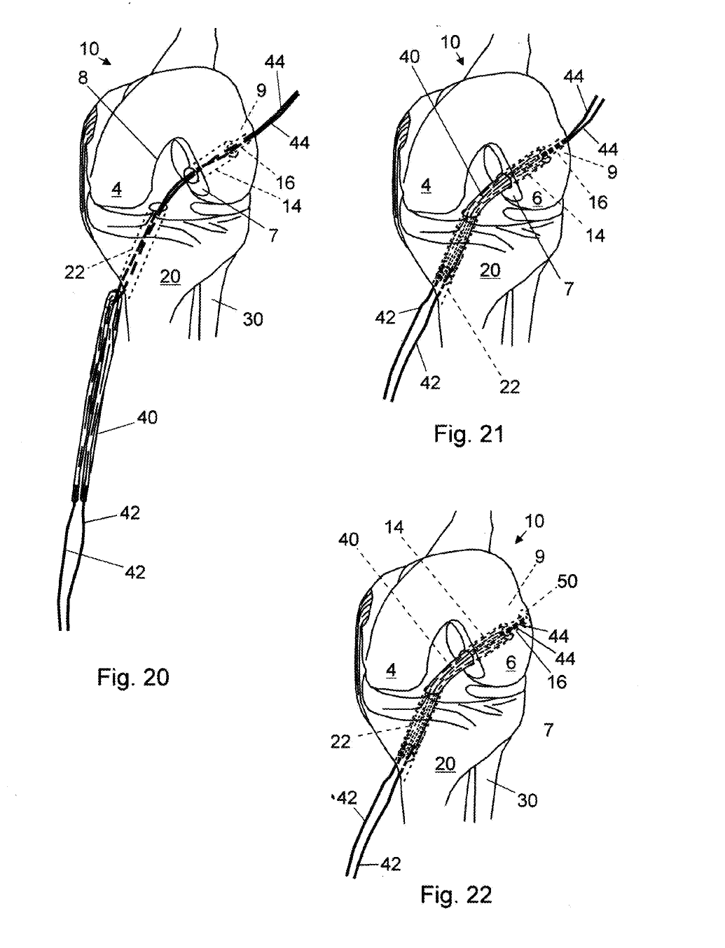

FIG. 20 depicts a soft tissue graft 40 prepared in accordance with standard procedures for placement in the knee. Distal sutures 44 have been threaded through tibial tunnel 22, femoral tunnel 14 and hole 16 to exterior lateral surface 9 of femur 10 so as to allow graft 40 to be drawn into position as depicted in FIG. 21, and fixed therein using button 50 to which distal sutures 44 are affixed positioned against lateral surface 9 of femur 10 (see FIG. 22). Thereafter graft 40 is tensioned and affixed in tibial tunnel 22 by an interference screw or button fixation using standard techniques and devices.

In the foregoing example, femoral tunnel 14 is formed in an anatomic position using drilling devices 200 and 300 having rigidly angularly offset distal portions 201 and 301 respectively that allow access without interference from medial condyle 4 and without hyperflexing the knee. When a femoral tunnel is formed using prior art methods, using the antero-medial portal and hyperflexing of the knee, together with rigid linear drilling and reaming devices, the tunnel length is limited by the relative angle between the drilling device and the lateral condyle. By using drilling devices with angularly offset distal portions and the associated methods of the current invention, the relative angle between femoral tunnel 14 and the lateral condyle 6 may be increased with an associated increase in the tunnel length.

Other prior art methods for forming a femoral tunnel from the “inside” use flexible guide pins placed from either the inside or outside, and flexible reamers mounted into standard powered drill handpieces that follow the guide pin and are introduced via the antero-femoral portal. In a preferred embodiment, the method of the present invention uses one or more simple single-use rigid drilling devices that may be mounted in a standard arthroscopy shaver handpiece. This eliminates the need to clean and sterilize the flexible drills and reamers between cases.

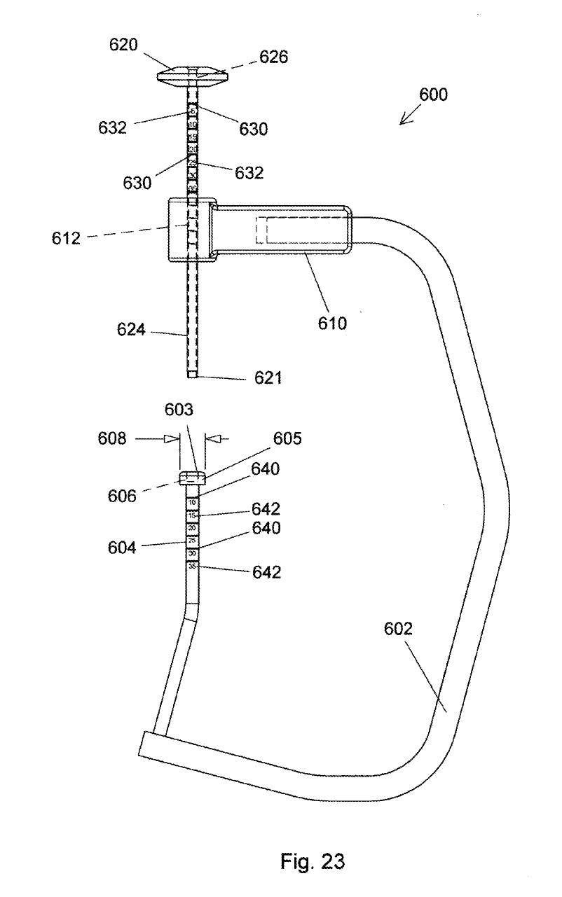

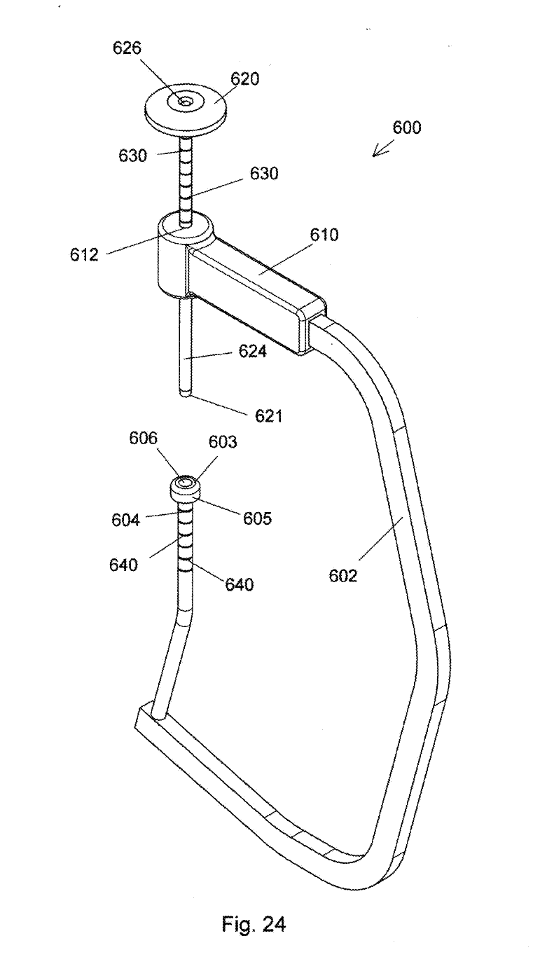

FIGS. 23 and 24 depict a drill guide 600 contemplated by the present invention for use in an alternate embodiment method for forming a femoral tunnel from the inside out for suspensory fixation. Drill guide 600 has a frame 602 with a distally mounted portion 604 having a proximal locating portion 605 of diameter 608. In the proximal surface 603 of proximal locating portion 605 is formed cylindrical recess 606. Frame 602 has a proximal portion 610 in which is formed, coaxial with proximal locating portion 605, cylindrical cannulation 612. Slidably positioned within cannulation 612 is an elongate distal portion 624 of proximal guide element 620. Lumen 626, sized to receive a guide-pin therein, extends the length of proximal guide element 620. Distal portion 604 has formed thereon graduations 640 and indicia 642 indicating the distance from the proximal face 603 of proximal locating portion 605 of distal portion 604. Proximal guide element 620 has formed thereon graduations 630 and indicia 632 that indicate the distance from the proximal face 603 of proximal locating portion 605 to the distal end 621 of elongate portion 624. Drill guide 600 as depicted diameter 608 of proximal locating portion 605 is greater than the diameter of other portions of locating portion 605. In other embodiments, diameter 608 is reduced to increase the ease of placement and positioning of locating portion 605 in a femoral tunnel during use.

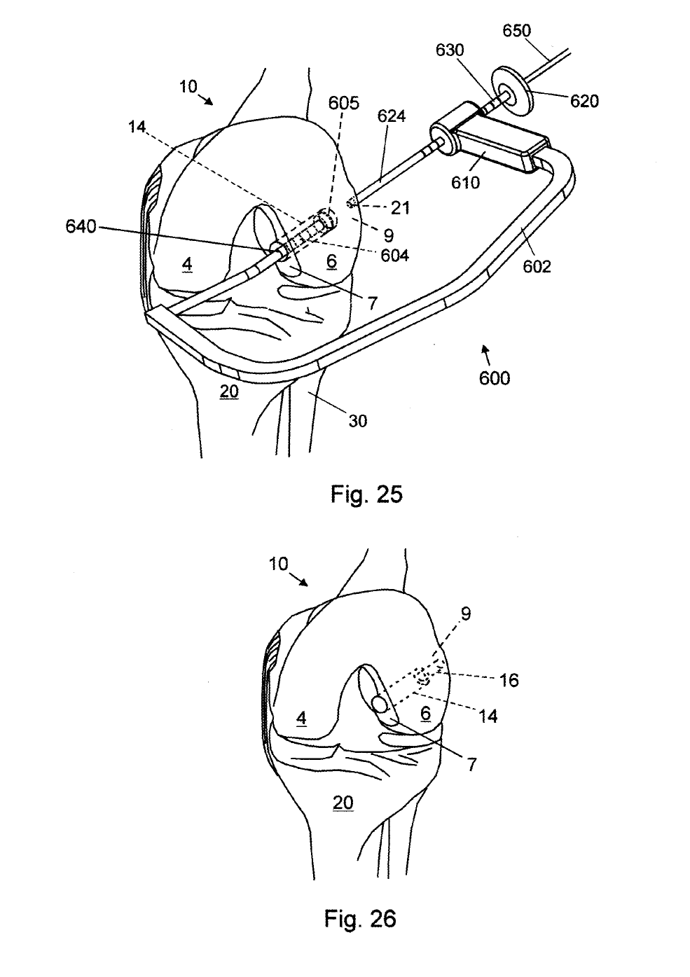

The use of drill guide 600 in an alternate method of forming a femoral tunnel for suspensory fixation is depicted in FIG. 25. Tunnel 14 is formed in the manner previously herein described. Thereafter, using the antero-medial portal, distal portion 604 of drill guide 600 is positioned within socket 14 as shown, proximal face 603 of proximal portion 605 of distal portion 604 contacting the end of the socket 14 as depicted. Distal end 621 of elongate distal portion 624 of proximal guide element 620 is positioned against lateral surface 9 of the femur at the desired location for the proximal end of hole 16 (see FIG. 18) to be formed using drill guide 600 and a drill tip guide pin 650. Graduations 640 and indicia 642 on distal portion 604 of guide 600 indicate for the surgeon the length of tunnel 14. Graduations 630 and indicia 632 on elongate portion 624 of proximal guide element 620 indicate the distance from the end of tunnel 14 to the distal end 621 of guide element 620. At the completion of drilling with guide pin 650, the knee is as depicted in FIG. 26, which corresponds to the knee condition as depicted in FIG. 18, and subsequent steps for placement of a tissue graft are as previously herein described and depicted in FIGS. 20 through 22.

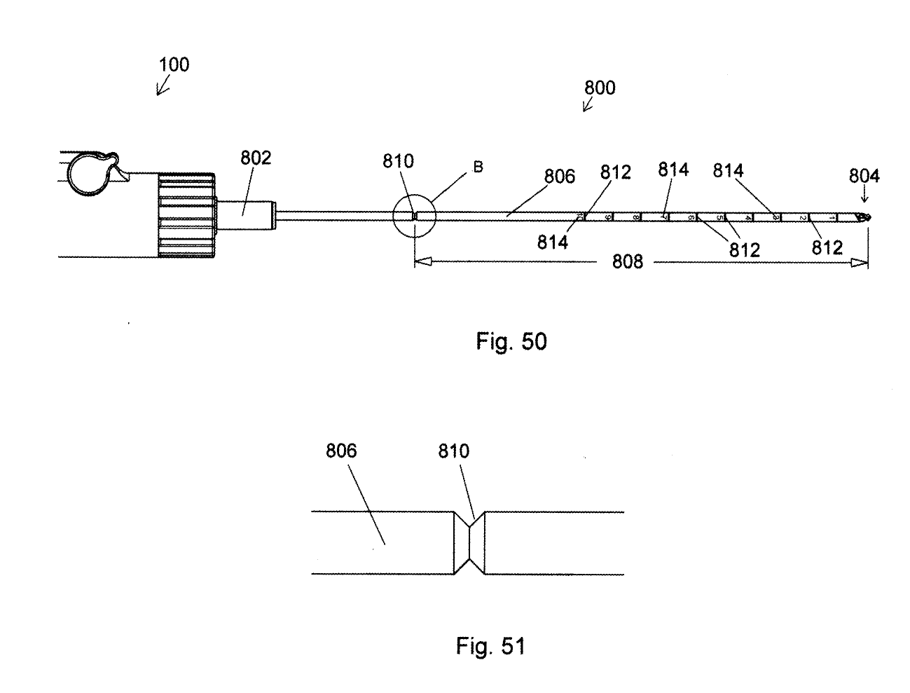

Drill tip guide pin 650 may be provided with a standard configuration adapted to mate with and be powered by a conventional drill handpiece, or in an alternatively preferred embodiment, may be powered by an arthroscopy shaver handpiece. FIGS. 50 and 51 depict a drill tip guide pin 800 of the present invention (corresponding to drill tip guide pin 650 in FIG. 25) having a proximal hub assembly 802 removably mounted to handpiece 100, a distal drill portion 804, and an elongate portion 806 therebetween. Guide pin 800 is used in the same manner as prior art drill tip guide pins except that guide pin 800 is driven by shaver handpiece 100 rather than a standard orthopedic powered drilling device.

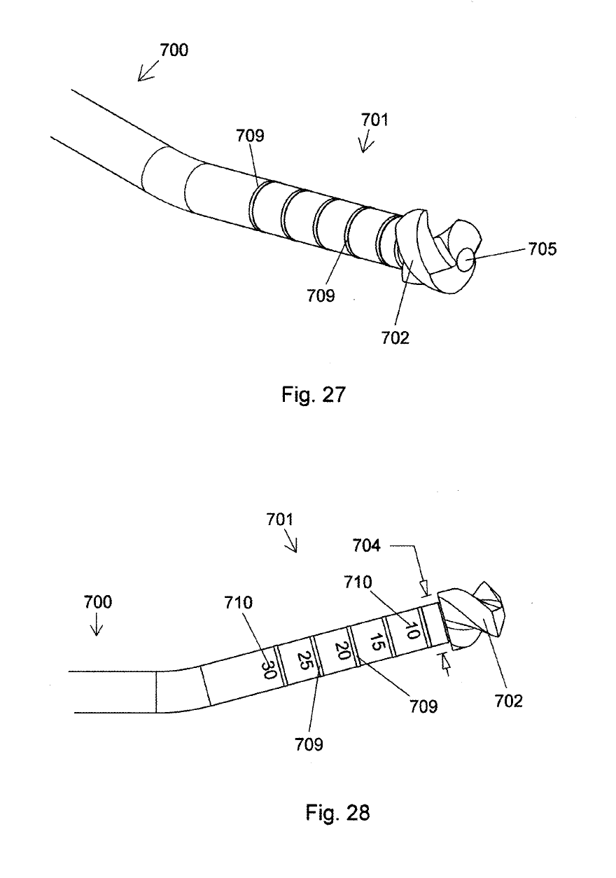

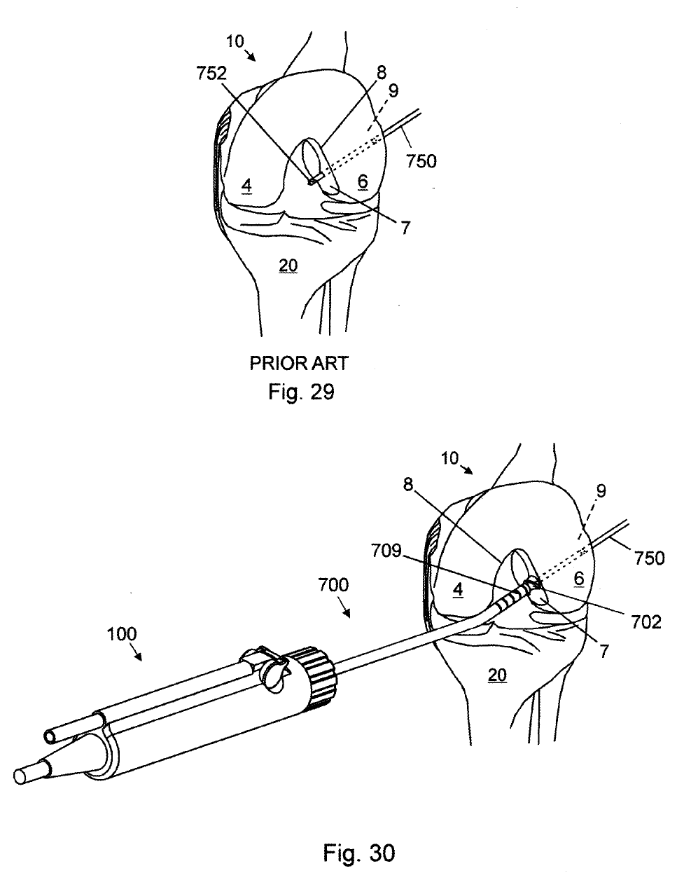

FIGS. 27 and 28 depict the distal portion of drilling device 700 which is alike in all aspects of form and function to drilling device 300 (FIGS. 4 through 6) except as subsequently specifically described. Specifically, cutting element 702 has formed therein cannulation 705 that is sized to slidably and rotatably receive a guide pin such as described above (not shown). Reduced diameter distal portion 303 of cutting element 302 (FIG. 6) is eliminated. Distal portion 701 has formed thereon graduations 709 and indicia 710 indicating the distance from the distal end of cutting element 702.

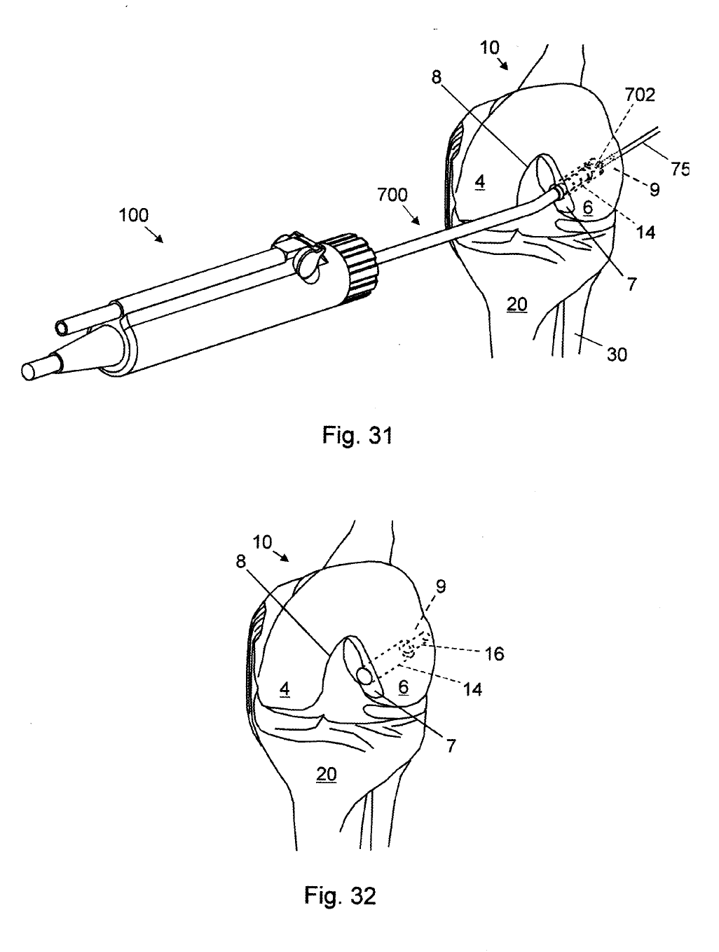

In an alternate embodiment, the present invention provides a method for forming a femoral tunnel for suspensory fixation of a soft tissue graft placed therein, wherein a drill tip guide pin 750 is placed from the outside using prior art techniques incorporating a conventional aimer. In a preferred embodiment, drill tip guide pin 750 is like guide pin 800 (FIGS. 50 and 51) of the present invention, the use of which is subsequently described in detail with regard to forming a tibial tunnel. Thereafter, a drilling device 700 having a cutting element 702 with a suitable diameter 704 is selected and mounted in shaver handpiece 100. Drilling device 700 is inserted into the knee via the anterolateral portal, cannulation 705 engaging the end of drill end guide pin 750 so as to establish coaxial alignment between distal portion 701 of drilling device 700 and guide pin 750. Thereafter handpiece 100 is activated and drill 700 advanced coaxial to guide pin 750 (see FIG. 31) until the desired tunnel depth is attained as indicated by graduations 709 and indicia 710 (FIG. 28). Drilling device 700 is then withdrawn leaving the femur as depicted in FIG. 32, which corresponds to the femur as depicted in FIG. 18, and subsequent steps for placement of a tissue graft according to the principles of the present invention are as previously herein described.

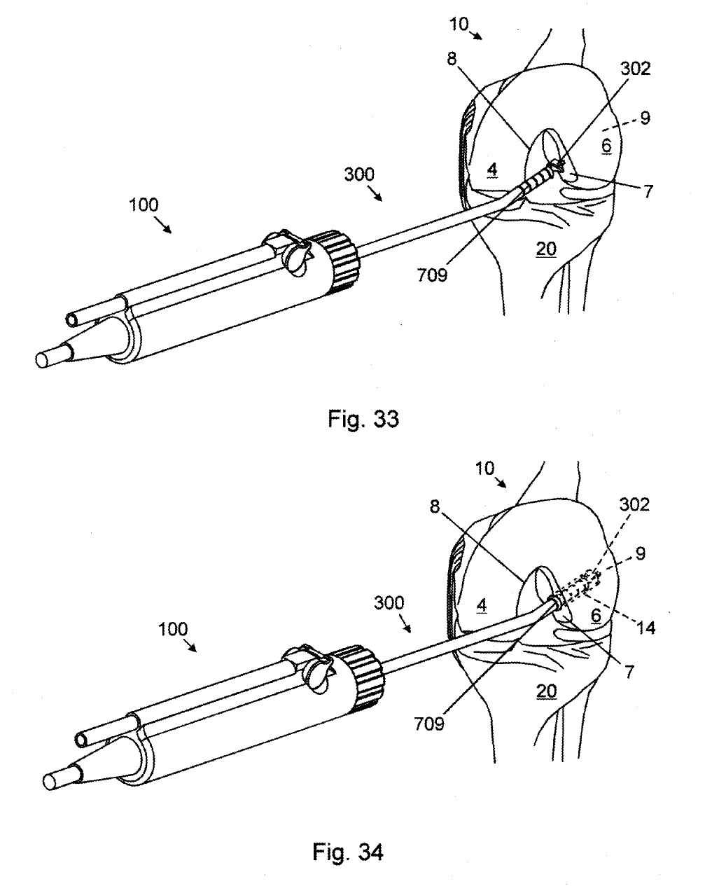

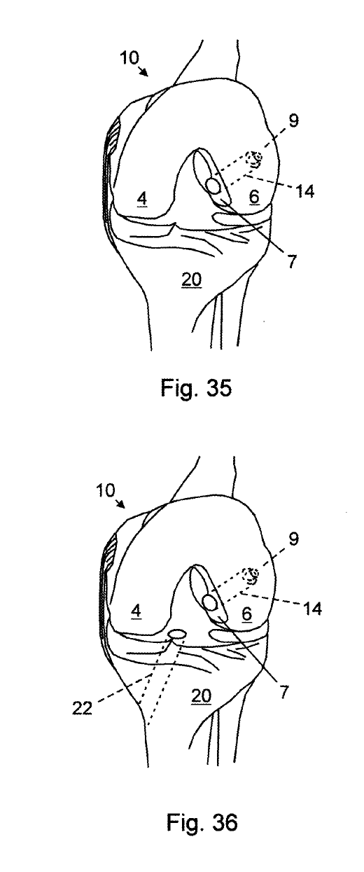

While the previous embodiments describe suspensory fixation using a button or other suitable suspensory fixation device, the present invention contemplates alternate embodiments in which aperture fixation is used, for example using an interference screw. FIGS. 33 through 41 depict such a method for femoral fixation using an interference screw in accordance with the present invention. As shown in FIG. 33, drilling device 300 is mounted in shaver handpiece 100 and then inserted into the knee as previously described, with the distal end of reduced portion 303 of cutting element 302 (see FIG. 6) positioned at the anatomic location for the tunnel placement. Shaver handpiece 100 is activated and tunnel 14 drilled as shown in FIG. 34 to produce a tunnel 14 as shown in FIG. 35. Subsequently, tibial tunnel 22 may be formed as depicted in FIG. 36 using conventional prior art techniques.

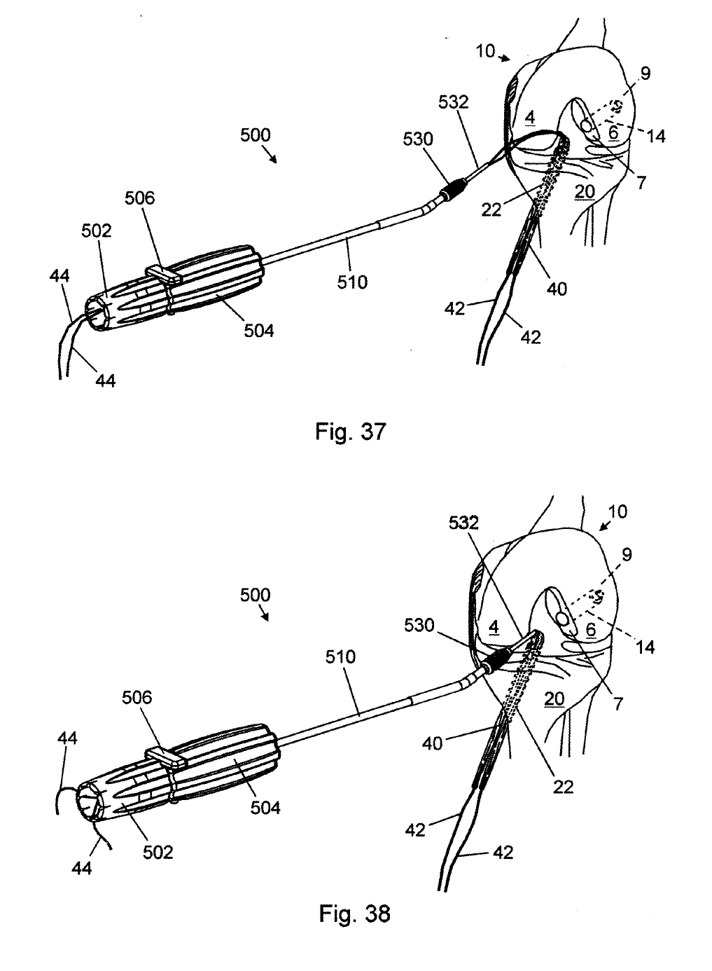

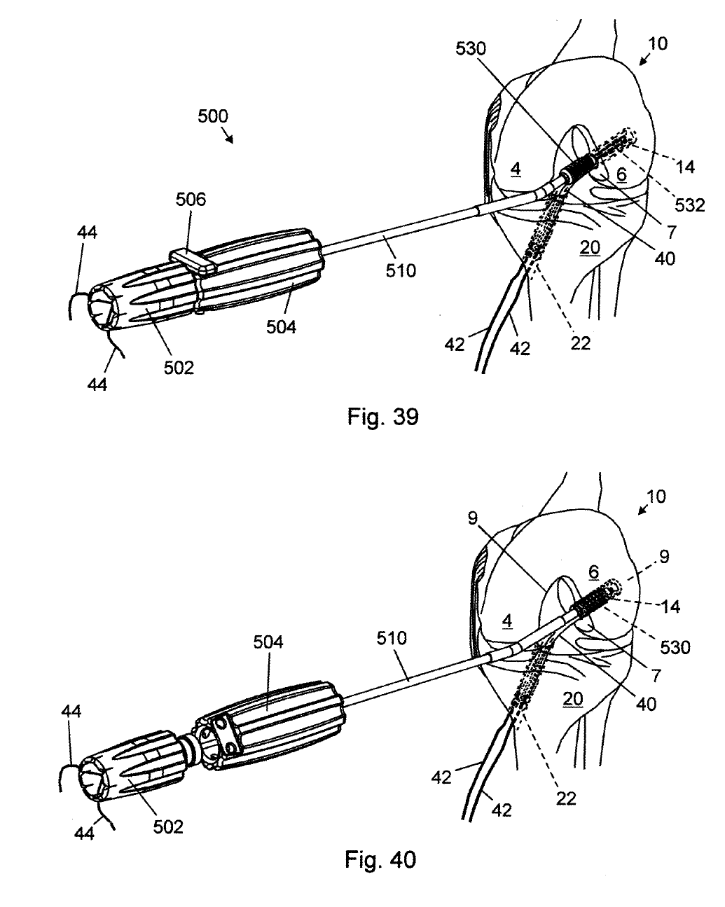

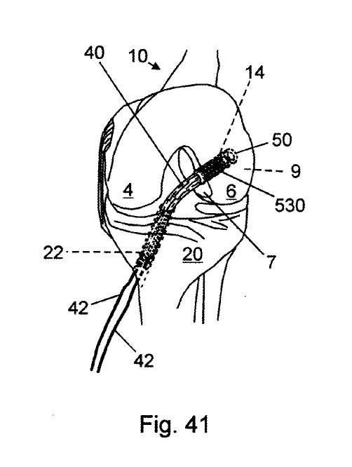

Distal sutures 44 are then used to pull graft 40 into tibial tunnel 22. As shown in FIG. 37, distal sutures 44 of graft 40 are drawn into tubular distal element 532 of implant placement system 500 using a loading loop (not shown) and therefrom through a central cannulation of the device such that the ends of distal sutures 44 extend from the proximal end of handle 502. Thereafter, graft 40 may be releasably positioned at the distal end of distal element 532 by pulling on distal sutures 44 extending from the proximal end of handle 502, and maintained in that position by optionally cleating sutures 44 disposed at the proximal end of handle 502 such as depicted in FIG. 38. The distal end of the graft is positioned within femoral tunnel 14 by inserting distal element 532 of implant placement system 500 into tunnel 14 as shown in FIG. 39. Thereafter, driver handle 504 is released from tensioning handle 502 (in this case by removing key 506) and interference screw 530 is threaded into tunnel 14 so as to affix the distal end of graft 40 in tunnel 14. Sutures 44 are then released from the cleats on tensioning handle 502 and implant system 500 removed from the site. Distal sutures 44 are then trimmed resulting in the femoral fixation depicted in FIG. 41. Thereafter, graft 40 is tensioned and affixed in tibial tunnel 22 by means of an interference screw or button fixation using standard techniques and devices that are not considered part of the present invention.

In the tibial fixation method by interference screw as herein described, the distal portion of graft 40 is “whip stitched”, in that multiple circumferential sutures are placed on the portion of the graft to be inserted into socket 14 and secured by the implant 530. This stitching increases the pull-out strength of the finished construct and can minimize possible slippage of graft 40 within femoral tunnel 14 when the graft 40 is under load.

In certain cases, it is desirable to use a bone-tendon-bone (“BTB”) graft, wherein the graft has distal and proximal bone portions (“bone plugs”) that are attached in the femoral and tibial tunnels. Femoral fixation methods according to the principles of the present invention are subsequently described.

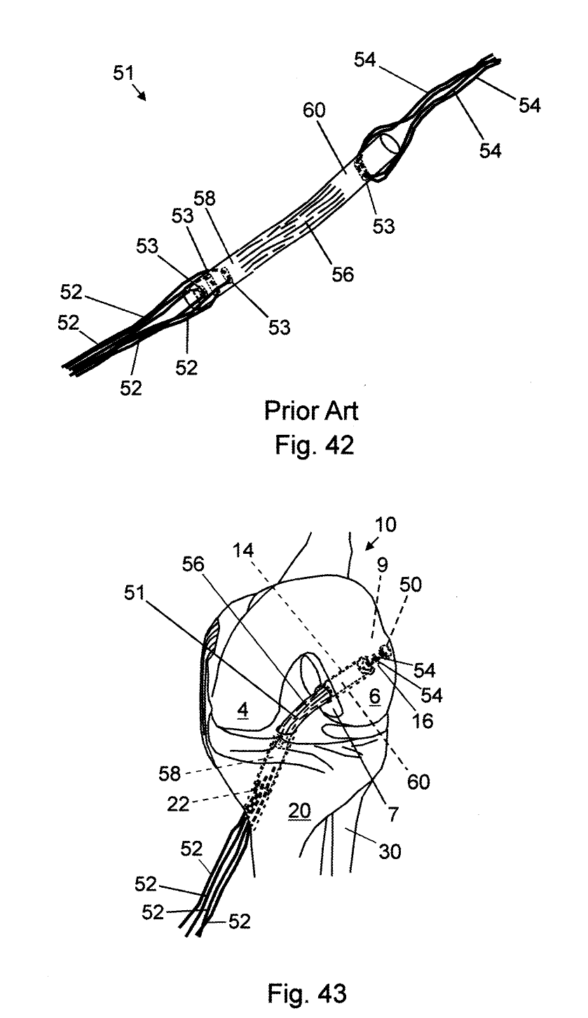

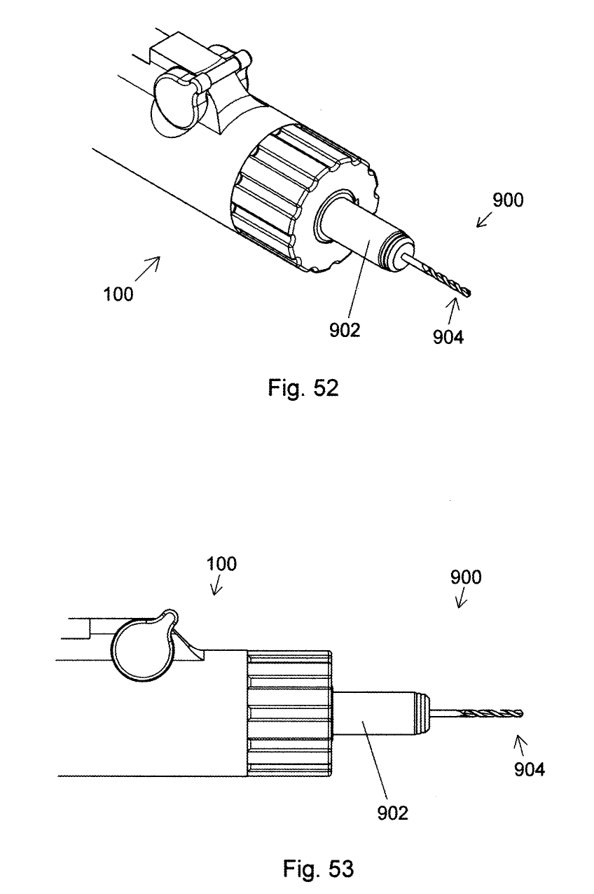

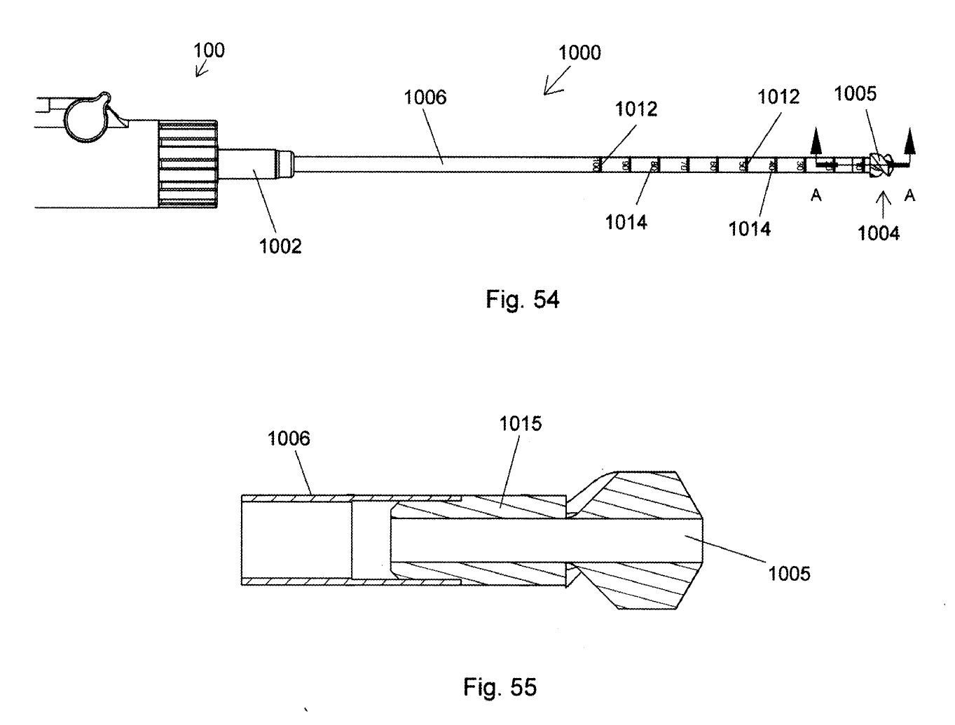

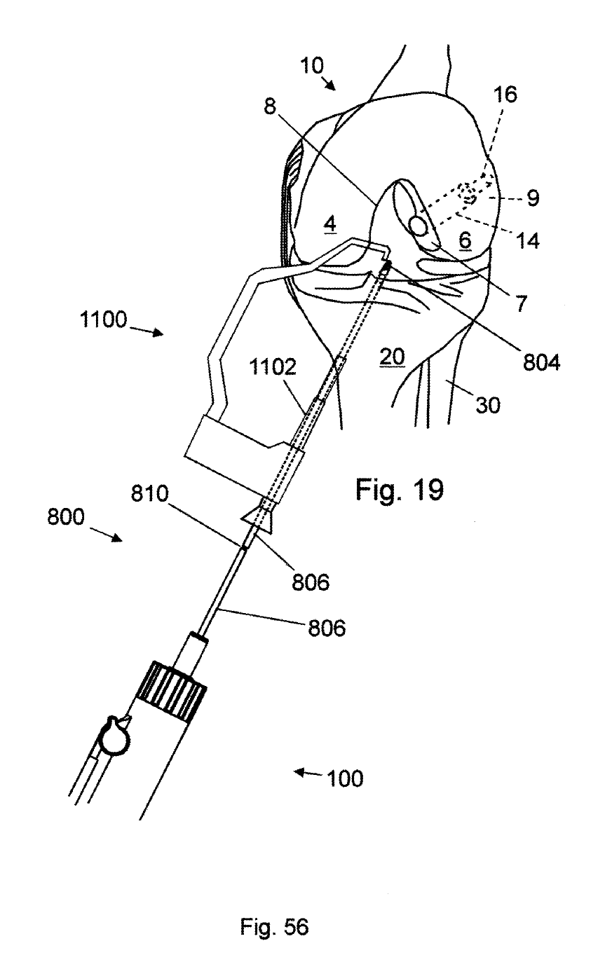

Referring to FIG. 42, BTB graft 51 includes a tendon 56, a distal bone plug 60 to which is attached distal sutures 54, and a proximal bone plug 58 to which is attached proximal sutures 52. Sutures 52 and 54 are attached to the bone plugs using drilled holes 53. In a preferred embodiment, holes 53 are formed using a drilling device 900 powered by a shaver handpiece 100 as depicted in FIGS. 52 and 53. Drilling device 900 has a proximal hub assembly 902 that allows for releasable mounting in shaver handpiece 100, and an elongate distal drilling portion 904. In other embodiments, conventional drilling devices may be used. As depicted in FIG. 43, BTB graft 51 may be positioned and affixed using button 50 or another suitable suspensory fixation device using the methods and devices previously herein described. Alternatively, according to the principles of the present invention in an alternate embodiment, BTB graft 51 may be affixed in the femoral tunnel using an interference screw placed using devices and methods of the present invention.

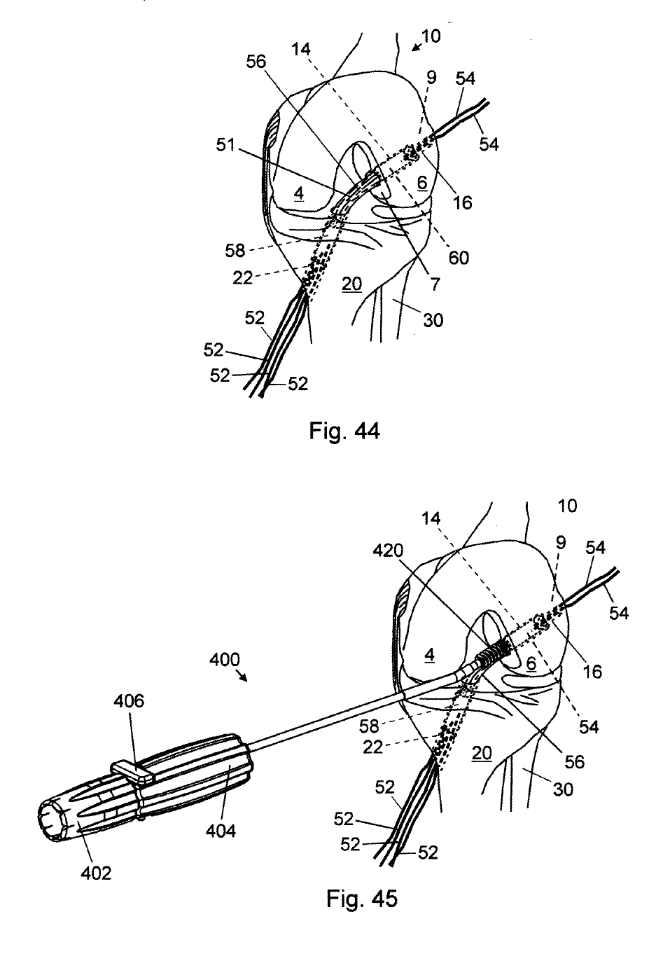

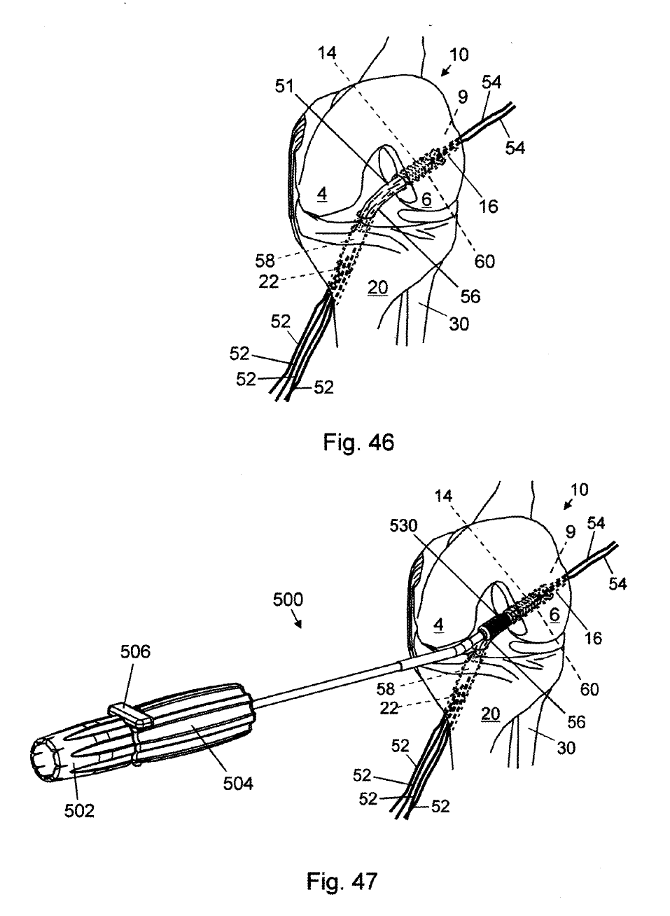

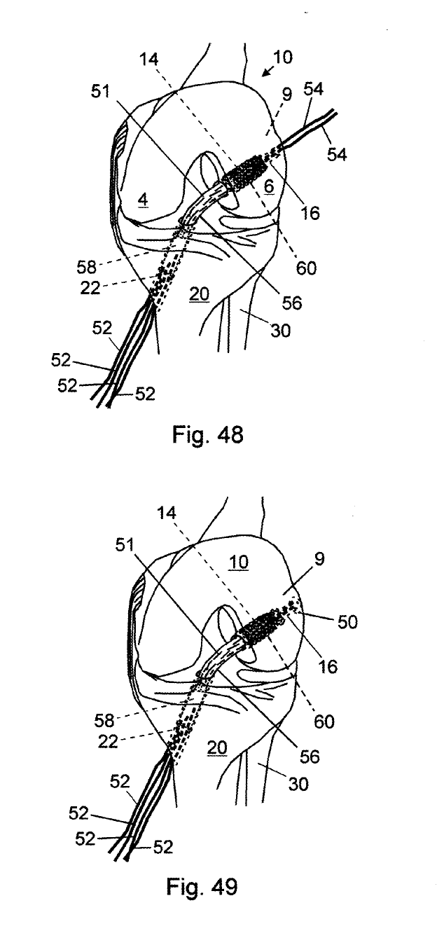

In FIG. 44, graft 51 has been positioned for fixation as previously herein described and depicted in FIGS. 13 through 21. Thereafter, as shown in FIG. 45, endoscopic tapping device 400 is inserted via the anteromedial portal and threading element 420 is positioned at the location selected for placement of the interference screw. By rotating handle 404, torque is supplied to threading element 420 so as to produce a threaded socket between tunnel 14 and distal bone plug 60 as depicted in FIG. 46. Subsequently, implant placement system 500 is inserted into the knee as depicted in FIG. 47, with distal element 532 being positioned within the threaded socket previously formed by tapping device 400. Thereafter, handle 504 is uncoupled from handle 502, in this case by removing key 506, thereby allowing handle 504 and its associated drive elements to supply torque to interference screw 530 so as to thread implant 530 into the prepared socket. FIG. 48 depicts graft 51 positioned in the knee with femoral fixation supplied by interference screw 530 positioned in the threaded socket formed by tapping device 400.

In FIG. 49 this interference screw fixation is “backed up” by button 50 to give secure fixation through both suspensory and aperture means.

As noted previously, FIGS. 50 and 51 depict a drill end guide pin 800 that may be removably mounted in shaver handpiece 100. Elongate distal portion 806 has at its distal end a distal drilling portion 804 and at its proximal end a proximal end hub assembly 802 configured for removable mounting to shaver handpiece 100, and. Proximal to the distal end of elongate portion 806, at a distance 808, circumferential notch 810 is formed. Graduations 812 and 814 indicate the distance from the distal end of elongate portion 806. Drill tip guide pin 800 is configured such that after guide pin 800 is placed by drilling, elongate distal portion 806 may be fractured at circumferential notch 810 so as to allow for removal of any drilling guides used during placement of guide pin 800. Subsequently, guide wire 800 may be applied in the same manner as prior art guide pins, placed by conventional means.

FIGS. 52 and 53 depict a drilling device 900 of the present invention that finds utility in forming holes 53 in bone plugs 58 and 60 (see FIG. 42) of a BTB graft. Drilling device 900 has a proximal hub assembly 902 that enables removable mounting to shaver handpiece 100, and a distal drilling portion 904 having a diameter and length suitable for forming holes 53.

Cannuluated drilling device 1000, also referred to as a “cannulated reamer”, is configured for forming tunnels for ACL and PCL repair in methods of the present invention. Drilling device 1000 has an elongate tubular distal portion 1006 having at its proximal end hub assembly 1002 configured for removable mounting to shaver handpiece 100. Elongate tubular distal portion 1006 has mounted to its distal end cutting element 1015 with cannulation 1005. Cutting element 1015 is secured to the distal end of tubular distal element 1006 by laser welding, brazing, mechanical or other suitable securing means. Graduations 1012 and indicia 1014 indicate the distance from the distal end of cutting element 1015.

The method of forming tibial tunnels in accordance with the principles of the present invention differs from methods of the present invention for producing femoral tunnels in that tibial tunnels are formed from the outside (as opposed to the inside) and use rigid linear (as opposed to angularly offset) devices. Specifically, conventional prior art drilling fixtures may be used to locate the tunnel in a standard manner. Thereafter, drill tip guide pin 800 is used to establish the tunnel path and cannulated drilling device (reamer) 1000 is used to form the tunnel. However, unlike prior art methods for forming tibial tunnels, the devices of the present invention are configured to be powered by shaver handpiece 100 as opposed to a conventional power drilling system.

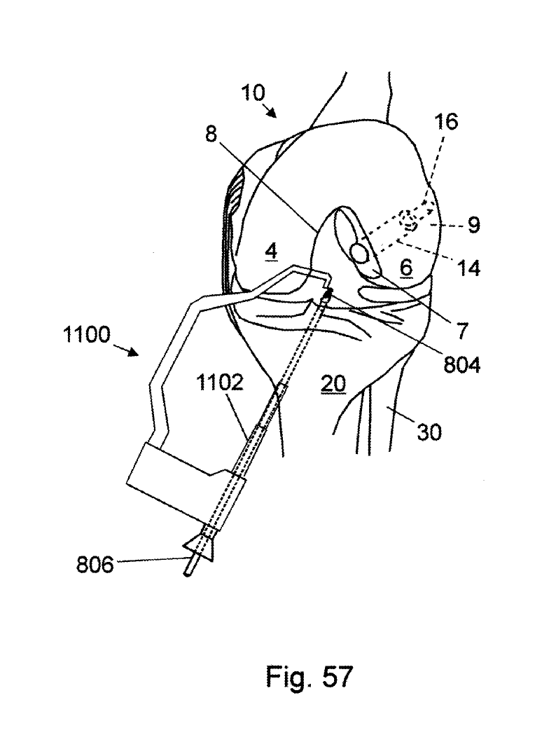

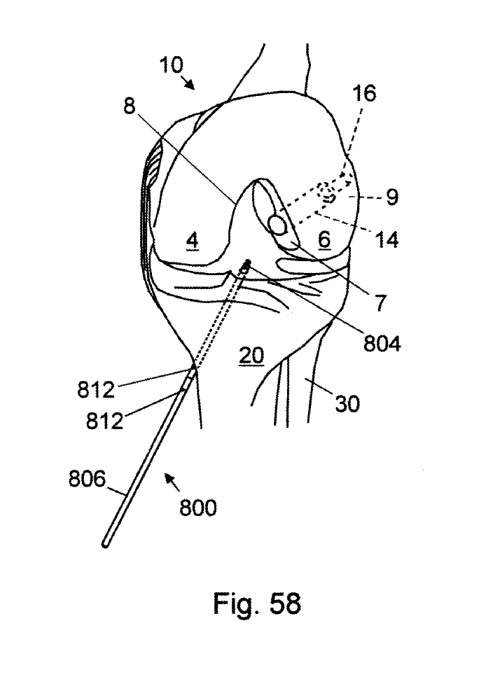

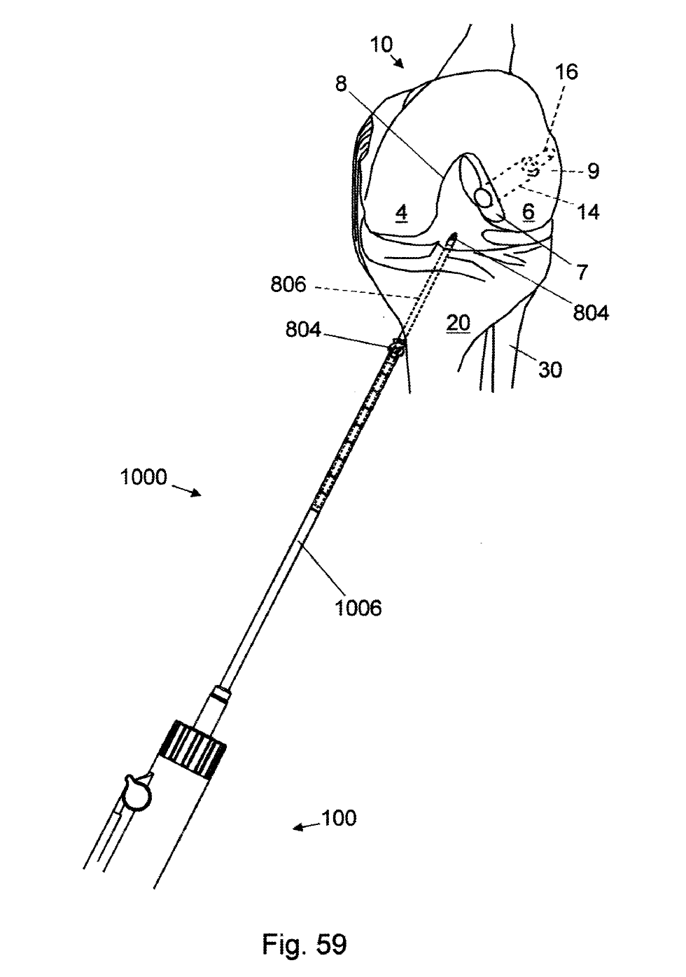

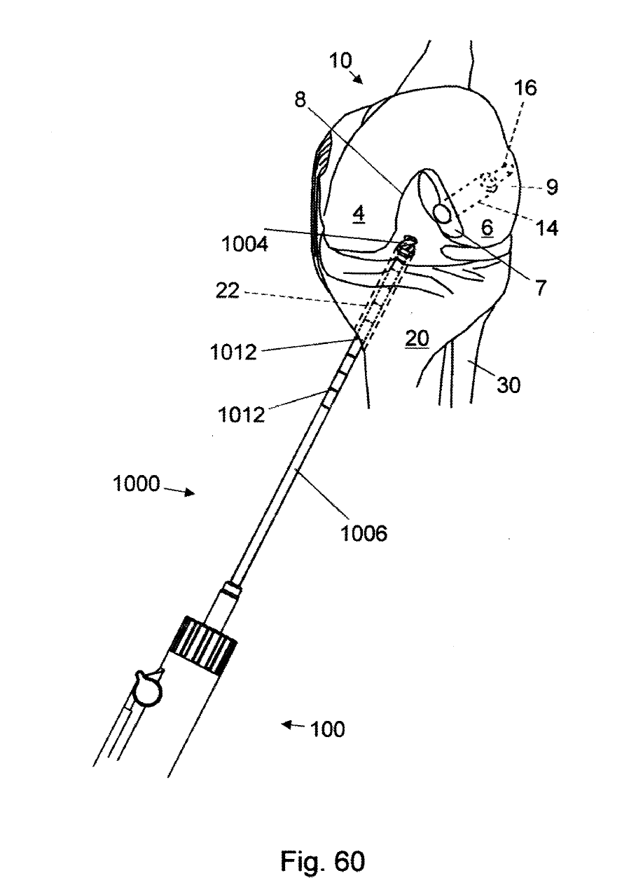

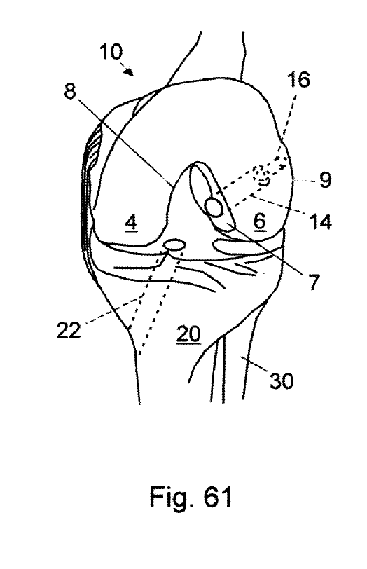

Referring now to FIG. 56, prior art drill guide 1100 is positioned with the distal end at the anatomic location for the graft, and cannulated proximal portion 1102 establishing a path thereto. Using power from shaver handpiece 100, drill tip guide pin 800 (see FIGS. 50 and 51) is placed with distal end 804, passing from the tibial plateau at the selected location. Thereafter, elongate distal portion 806 of guide pin 800 is fractured at circumferential notch 810 as shown in FIG. 57. Drill guide 1100 is then removed leaving guide pin 800 in position as depicted in FIG. 58. The length of the tunnel is determined using graduations 812 and indicia 814 (FIG. 50). Referring now to FIG. 59, cannulated drilling device 1000 mounted in shaver handpiece 100 is positioned as depicted with elongate distal portion 806 of guide pin 800 positioned within cannulation 1005 of distal cutting element 1015 of drilling device 1000 so as to establish the path for forming the tibial tunnel. Handpiece 100 is subsequently activated and cannulated reamer 1000 advanced to the position depicted in FIG. 60. Because the surgeon knows the tunnel length from the graduations on drill tip guide pin 800, forward force on reamer 1000 can be decreased as distal cutting element 1015 approaches the tibial plateau so as to prevent damage to femoral condyle 6. Thereafter, the knee is as depicted in FIG. 61 and placement, fixation and tensioning are as previously herein described.

Methods for forming femoral and tibial tunnels in accordance with the present invention make extensive use of single-use devices. These may include drilling devices 200, 300, 700, and 1000, and drill tip guide pin 800. In another aspect of the present invention, the single-use devices required for forming the tunnels for an ACL or PCL replacement may be supplied as a kit, sterile and ready for use. Optionally, devices required for suspensory or aperture fixation and systems for their placement may included in the kits also.

### INDUSTRIAL APPLICABILITY

As noted previously, there is a need in the art for simple, reliable methods for forming femoral and tibial sockets or tunnels for graft fixation, such as in connection with ACL and PCL repair and reconstruction, that use single-use disposable devices and allow precise anatomical positioning of the tunnel. The present invention addresses this need by providing unique distal end cutting elements and angularly offset endoscopic drilling devices that are specifically configured for use with a conventional shaver handpiece and that are further optionally provided with guidance indicia and depth markings that enable tunnel formation in remote and difficult to access boney surfaces using minimally invasive procedures and graft placement using aperture and/or suspensory fixation means. Although described in detail with respect to ligament repair, particularly reconstruction of a ruptured ACL or PCL, it will be readily apparent to the skilled artisan that the utility of the present invention may be extended and/or adapted to other tissues and injuries.

The disclosure of each publication, patent or patent application mentioned in this specification is specifically incorporated by reference herein in its entirety. However, nothing herein is to be construed as an admission that the invention is not entitled to antedate such disclosure by virtue of prior invention.

The invention has been illustrated by reference to specific examples and preferred embodiments. However, it should be understood that the invention is intended not to be limited by the foregoing description, but to be defined by the appended claims and their equivalents.

What is claimed:

1. A method for preparing a human subject for reconstructive graft surgery of the knee, said method comprising steps of:
a. providing an endoscopic shaver handpiece housing a drive motor;
b. providing a first endoscopic drilling device having a proximal end configured to removably attach to a distal end of said endoscopic shaver handpiece, an elongate middle portion defining the longitudinal axis of the drilling device, a distal portion that is angularly offset from the longitudinal axis of the elongate middle portion, and a rotatable cutting element disposed at a distal end of said distal portion;
c. mounting the proximal end of said first endoscopic drilling device to the distal end of said endoscopic shaver handpiece;
d. inserting said first endoscopic drilling device into the subject's knee via a portal of the knee and positioning the distal end cutting element at an anatomical location of interest selected for interior femoral socket or tunnel formation on a surface of the medial or lateral condyle of the subject's femur, whereby the angular offset of the distal portion of the drilling device allows said device to reach around the medial or lateral condyle; and
e. activating said handpiece to provide rotational energy to said rotatable cutting element and thereby drill an interior socket of pre-determined depth in the medial or lateral condyle of the femur.

2. The method of claim 1, wherein said portal is a primary portal selected from the group consisting of the anterolateral, anteromedial, superomedial, and superolateral portals.

3. The method of claim 1, wherein said portal is a secondary portal selected from the group consisting of the posteromedial and posterolateral portals.

4. The method of claim 1, wherein said reconstructive graft surgery is anterior cruciate ligament reconstruction and said interior socket is formed in the lateral condyle of the femur.

5. The method of claim 1, wherein said reconstructive graft surgery is posterior cruciate ligament reconstruction and said interior socket is formed in the medial condyle of the femur.

6. The method of claim 1, wherein said anatomical location of interest is identified prior to surgery and marked by the surgeon by means of an awl or other impacting device, wherein said awl or other impacting device is used to form a conical depression in the condyle surface.

7. The method of claim 1, wherein the angle formed between the distal portion and the middle portion of said first endoscopic drilling device ranges from 2 to 40 degrees.

8. The method of claim 1, wherein the distal portion of said first endoscopic drilling device is provided with a plurality depth markings and associated numeric indicia.

9. The method of claim 1, wherein a cannulated implant for aperture fixation of a reconstructive tissue graft is inserted into said interior socket.

10. The method of claim 9, wherein said cannulated implant comprises a threaded anchor or interference screw.

11. The method of claim 1, further comprising the step (f) in which the depth of the interior socket is extended so as to form a through-and-through tunnel to the medial or lateral surface of the femur.

12. The method of claim 11, further wherein sutures affixed to a reconstruction tissue graft are used to pull the graft into position and secure it in said through-and-through femoral tunnel by means of suspensory fixation.

13. The method of claim 12, wherein said suspensory fixation comprises button fixation.

14. The method of claim 11, wherein said step (f) is performed using a second endoscopic drilling device, wherein:
a. said second endoscopic drilling device has a proximal end that is affixed to the distal end of said endoscopic shaver handpiece, an elongate middle portion defining the longitudinal axis of the second endoscopic drilling device, a distal portion that is angularly offset from the longitudinal axis of the elongate middle portion, and a rotatable cutting element disposed at a distal end of said distal portion,
b. the angular offset of said second endoscopic drilling device is equal to the angular offset of said first endoscopic drilling device, and
c. a diameter of distal cutting element of said of said second endoscopic drilling is less than a diameter of the distal cutting element of said first endoscopic drilling device.

15. The method of claim 14, wherein the diameter of distal cutting element of said of said second endoscopic drilling ranges from 2 to 13 mm and the diameter of the distal cutting element of said first endoscopic drilling device ranges from 6 to 16 mm.

16. The method of claim 11, wherein said step (f) is performed using a first endoscopic drilling device.

17. The method of claim 11, wherein said step (f) is performed using a drill guide comprising a curved frame having opposingly faced upper and lower ends, wherein:
a. said lower end is provided with an elongate transverse locating element having a diameter that permits said element to be slidably received within said interior socket;
b. said upper end is provided with cylindrical cannulation having an elongate guide element slidably received therein, wherein said guide element includes a lumen sized to receive a drill tip guide pin;
c. wherein the distal end of said guide element is faces and is co-linear with the distal end of said locating element.

18. The method of claim 17, wherein said through-and-through tunnel to the surface of the femur is formed by:
a. slidably inserting said elongate transverse locating element into said interior socket;
b. positioning the distal end of said guide element against the surface of the femur at the desired location for the proximal end of the femoral tunnel;
c. applying rotational force to said drill tip guide pin to form an exterior socket;
d. releasing the guide pin when the exterior socket meets the interior socket.

19. A method for reconstructing, repairing or replacing a ligament of a knee of a human subject using a soft tissue graft, said method comprising steps of:
a. inserting a first endoscopic drilling device affixed to an endoscopic shaver handpiece into the subject's knee via a portal of the knee, wherein said first endoscopic drilling device is characterized by an elongate middle portion defining the longitudinal axis of the drilling device, a distal portion that is angularly offset from the longitudinal axis of the elongate middle portion, and a rotatable cutting element disposed at a distal end of said distal portion;
b. positioning the offset distal end cutting element at an anatomical location of interest selected for interior femoral socket or tunnel formation on a surface of a medial or lateral condyle of the subject's femur, whereby the angular offset of the distal portion of the drilling device allows said device to reach around the medial or lateral condyle;
c. activating said handpiece to provide rotational energy to said rotatable cutting element and thereby drill an interior socket of pre-determined depth in the medial or lateral condyle of the femur;
d. providing a suitable socket or tunnel into an interior portion of the tibia; and
e. affixing one end of a reconstructive tissue graft to said femoral socket or tunnel and a second end of a reconstructive tissue graft to said tibial socket or tunnel.

20. The method of claim 19, wherein said ligament comprises the anterior cruciate ligament.

21. The method of claim 19, wherein said ligament comprises the posterior cruciate ligament.

22. The method of claim 19, wherein said method further comprises the step of extending the depth of the femoral socket or tunnel so as to form a through-and-through tunnel to the medial or lateral surface of the femur.

23. The method of claim 22, wherein the depth of said interior socket is extended using said first endoscopic drilling device.

24. The method of claim 22, wherein the depth of said interior socket is extended using a second endoscopic drilling device, wherein:
a. said second endoscopic drilling device has a proximal end that is affixed to the distal end of said endoscopic shaver handpiece, an elongate middle portion defining the longitudinal axis of the second endoscopic drilling device, a distal portion that is angularly offset from the longitudinal axis of the elongate middle portion, and a rotatable cutting element disposed at a distal end of said distal portion,
b. the angular offset of said second endoscopic drilling device is equal to the angular offset of said first endoscopic drilling device, and
c. a diameter of distal cutting element of said of said second endoscopic drilling is less than a diameter of the distal cutting element of said first endoscopic drilling device.

25. The method of claim 19, wherein step (a) is preceded by the following steps:
i. removing any damaged ligament tissue from the surgical site;
ii. selecting an anatomical location for the formation of an interior femoral socket or tunnel on a surface of a medial or lateral condyle of the subject's femur; and
iii. marking said selected anatomical location by forming a conical depression in a surface of said condyle using an awl or other impacting device.

26. A kit for reconstructing, repairing or replacing a damaged ligament of a knee of a human subject using a soft tissue graft, said kit comprising:
a. one or more elements for aperture fixation and/or suspensory fixation of a reconstructive tissue graft; and
b. a first endoscopic drilling device having a proximal end configured to removably attach to a distal end of an endoscopic shaver handpiece housing a drive motor, an elongate middle portion defining the longitudinal axis of the drilling device, a distal portion that is angularly offset from the longitudinal axis of the elongate middle portion, and a rotatable cutting element disposed at a distal end of said distal portion;

27. The kit of claim 26, further comprising a second endoscopic drilling device having a proximal end configured to removably attach to the distal end of said endoscopic shaver handpiece, an elongate middle portion defining the longitudinal axis of the second endoscopic drilling device, a distal portion that is angularly offset from the longitudinal axis of the elongate middle portion, and a rotatable cutting element disposed at a distal end of said distal portion, wherein the angular offset of said second endoscopic drilling device is equal to the angular offset of said first endoscopic drilling device, and a diameter of the distal cutting element of said second endoscopic drilling is less than a diameter of the distal cutting element of said first endoscopic drilling device.

28. The kit of claim 27, further comprising a drill guide comprising a curved frame having opposingly faced upper and lower ends, wherein:
a. said lower end is provided with an elongate transverse locating element having a diameter that permits said element to be slidably received within said interior socket;
b. said upper end is provided with cylindrical cannulation having an elongate guide element slidably received therein, wherein said guide element includes a lumen sized to receive a drill tip guide pin;
c. wherein the distal end of said guide element is faces and is co-linear with the distal end of said locating element.
